(19) United States
(12) Patent Application Publication
(10) Pub. No.: US 2005/0120395 A1
(43) Pub. Date: Jun. 2, 2005

(54) Method for genetically modifying a target population of an organism

(76) Inventor: Austin Burt, London (GB)

Correspondence Address: Ade & Company, 1700 360 Main Street, Winnipeg Manitoba, R3C 3Z3, CA

(21) Appl. No.: 10/497,390

(22) PCT Filed: Nov. 1, 2002
(86) PCT No.: PCT/GB02/04959

(30) Foreign Application Priority Data
Nov. 1, 2001 (GB) 0126251.8

(51) Int. Cl. C12Q 1/68; A01K 67/033; C07H 21/04; A01H 1/00; C12N 15/82; C12N 5/06; C12N 5/04

(52) U.S. Cl. 800/8; 800/278; 435/6; 435/69.1; 435/320.1; 435/348; 435/419; 536/23.5

(57) ABSTRACT

A method for genetically modifying a target population of an organism, comprising the steps of 1. providing a modified organism, wherein the modified organism is capable of sexually reproducing with an organism of the target population, and wherein a selected gene in the germline of the modified organism is disrupted by having inserted into it a sequence-specific nonMendelian selfish gene; 2. introducing the modified organism into the target population. A method for genetically modifying a target population of an organism, comprising the steps of 1. Introducing a homing endonuclease gene (HEG) into the germline of an organism which is capable of sexually reproducing with organisms of the target population; 2. introducing the modified organism into the target population. The HEG may encode a recombinant endonuclease, for example an endonuclease with a zinc-finger DNA binding domain and a sequence non-specific nuclease domain.

The present invention relates to population genetic engineering, including methods for transforming the gene pool of the population or species, for example for controlling the numbers of a population (population control) or for establishing a desired characteristic in the population.

There are two logically distinct situations in which one has to deal with genetically modified organisms (GMOs) in the environment. First, one might want to modify a population over which one has more-or-less complete control, for example a crop. In this case, one engineers in the lab whatever crop one wants, and then plants it out in a field. In terms of the environment, one is then concerned about minimising the effect of this on other species, and in particular in minimising the probability that the novel traits will spread through other species. This is mostly a problem in molecular biology; of engineering the right sort of organism. The probability of transfer can be reduced by, for example, making the plant sterile (or even just pollen sterile), or by having the novel trait discontinuously distributed in the genome (so no one part by itself would be functional); perhaps the ideal solution (which one day might be possible) is to make species (or organelles) with an alternative genetic code, and so the genes would not be functional in any other species that they might be transferred to.

The alternative situation is when one wants to modify a population over which one has little control, for example a pest. One might want to engineer a weed population, for example, so it cannot grow in farmers' fields, or engineer a rust or mildew population so it cannot attack a particular crop. This situation requires consideration of population biology in addition to molecular biology in order to achieve a genetically engineered population. This approach of modifying the pest as opposed to the crop has some advantages; for example, public worries about GMOs entering the human food chain can be put to rest.

For this latter sort of population genetic engineering, again one can imagine 2 scenarios. First, one might want to introduce a gene of interest into a population: for example, a gene with makes weeds susceptible to a herbicide, or a gene which makes mosquitoes unable to transmit malaria (see, for example Kidwell & Ribeiro (1992) Parasitol Toxicol 8, 325-329). The basic idea that has been proposed is to link the desired gene to a gene that is inherited at a greater than Mendelian rate (a nonMendelian gene e.g. a meiotic drive gene, or a transposable element, or a cytoplasmic incompatibility gene; see for example Hastings (1994) Phil Trans R Soc Lond B 344, 313-324), and have it drive the desired gene through the is population. This general but vague idea has been around since the 1970's, at least (see for example papers by Curtis referred to in Hastings (1994), for example Curtis et al (1976) Heredity 36, 11-29). However, it has never been used in practice. The main difficulties are in knowing what sort of nonMendelian genetic element or gene of interest to introduce, and how to prevent the spread of mutants in which the gene of interest has, for example, a stop codon in it. The most obvious genes one would want to introduce are harmful to the carrier and therefore selected against; a defective mutant (eg with a stop codon in the desired gene) would no longer impose a cost on the host, but would still have the nonMendelian transmission advantage, so it would drive even faster through the host population, preventing the harmful one from spreading.

Some forms of transposable elements have been suggested as suitable nonMendelian genetic elements in test systems, for example in Drosophila (see, for example, Hastings (1994) and Kidwell & Ribeiro (1992)), but even if such elements are known from the target species, they have disadvantages because there is no means of controlling the copy number or genomic location of insertion. Meiotic drive complexes are also unsatisfactory because there are very few known and they are unlikely to work if introduced into a new species. Wolbachia (endosymbiont bacteria which can give rise to cytoplasmic incompatibility, as discussed in, for example, Hastings (1994)) is not useful because it is not known how to engineer them.

LINEs are one of the 3 main classes of transposable elements (the other two are DNA transposons and LTR or retroviral-like retrotransposons). Many LINEs, including those known in humans, will insert almost anywhere in the genome, but there are some LINEs which are site-specific: they insert only into specific sequences. For example, R1 is a LINE in D. melanaogaster which inserts specifically at a particular nucleotide position in the 28S rRNA genes; R2 is another LINE which inserts at another position 74 bp upstream of R1. These elements are widespread among insects, having been described from the rRNA genes of 9 orders of insects. Site-specific LINEs have also been found in the pentanucleotide repeats at the telomeres of Bombyx mori and in spliced leader RNA (SL RNA) genes of trypanosomes and nematodes. In all cases known so far, the site-specific LINEs insert into multi-copy host genes (e.g., ribosomal genes), and so they can exist in multiple copies per haploid genome.

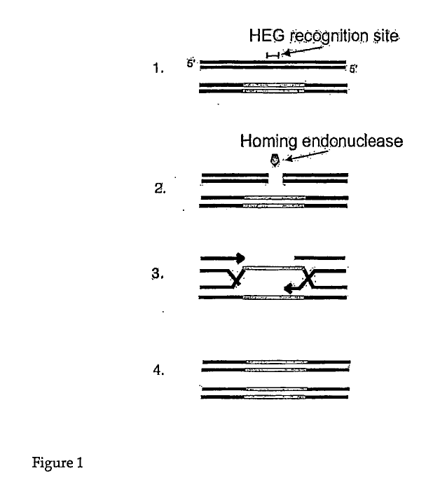

Homing endonuclease genes (HEGs) are optional or nonessential genes widely distributed within fungi, protists, bacteria, and viruses (Belfort & Roberts 1997; Mueller et al. 1993). At least among eukaryotes, they have no known host function, and instead are thought to be selfish or parasitic genes which spread in populations because their catalytic activity results in a biased pattern of inheritance (Hickey 1982). Any particular HEG exists only at one site in the genome, and codes for an enzyme which specifically recognises and cleaves sites not containing the gene. Thus, in heterozygous individuals, in which there are both HEG⁺ and HEG⁻ sites, the latter are cleaved by an enzyme made by the former. The cell then repairs the cut chromosome in the normal way, which involves using the intact HEG⁺ chromosome as a template for repair (Colaiacovo et al. 1999; Szostak et al. 1983). Thus, after repair, the heterozygote has been converted into an HEG⁺ homozygote (FIG. 1). Consequently, these genes show strong transmission ratio distortion, often being inherited by up to 95% of progeny, rather than the Mendelian 50%. A yeast HEG has been shown to be active in stimulating recombinational repair in Drosophila melanogaster (Bellaiche et al (1999) I-SceI endonuclease, a new tool for studying DNA double-strand break repair mechanisms in Drosophila. Genetics 152, 1037-1044; Rong & Golic (2000) Gene targeting by homologous recombination in Drosophila, Science 288, 2013-2018; Rong et al (2002) Targeted mutagenesis by homologous recombination in D. melanogaster. Genes Dev 16, 1568-1581).

The present invention provides an alternative method to introducing a desired gene into a population, in which a gene in a host population is disrupted (“knocked out”) using a selfish gene. This approach is considered to provide advantages in relation to instability in the face of nonfunctional mutations. The present invention also provides a sequence-specific nonMendelian selfish gene which may be designed for use in any target organism and which may be used for knocking out a gene in a host population or introducing a gene into a host population. These sequence-specific nonMendelian selfish genes are simpler to engineer, and may more readily and/or more rapidly be made with current technology, than (for example) meiotic drive genes or Wolbachia.

A first aspect of the invention provides a method for genetically modifying a target population of an organism, comprising the steps of
1. providing a modified organism, wherein the modified organism is capable of sexually reproducing with an organism of the target population, and wherein a selected gene in the germline of the modified organism is disrupted by having inserted into it a sequence-specific nonMendelian selfish gene;
2. introducing the modified organism into the target population.

By the term “gene” is included any portion of the genome the disruption of which leads to a difference in phenotype between an organism in which the portion is not disrupted (ie no copies of the portion are disrupted) and an organism in which both copies (or all copies, if there are more than two) of the portion are disrupted. There may also be a difference in phenotype between an organism in which the portion is not disrupted and an organism in which one or more, but not all, copies of the portion are disrupted.

It is preferred that the gene is a portion of the genome that is capable of being transcribed or an associated control region (for example an enhancer or promoter region). The gene may be transcribed to produce an RNA which (when suitably processed) encodes a polypeptide, or which forms a structural RNA. The gene may, for example, be a region that is necessary for correct chromosome segregation during meiosis, for example a cis-acting regions necessary for X-chromosome segregation at meiosis.

The gene may also be disrupted in the somatic tissue of the introduced organism. However, it is important that the introduced organism is able to reproduce. Thus, in the case of disruptions which have a recessive lethal or sterile phenotype, the introduced organisms are not homozygous in their somatic tissue (as they would be respectively not viable, or unable to pass on the gene disruption to progeny) as discussed further below. In order for the disruption to spread through the population from rare to common, individuals derived from heterozygous zygotes must be able to reproduce.

Clearly, for single-celled organisms, the single-cell is germ-line. For a single-cell organism (for example a malarial parasite (Plasmodium)) and gene disruptions which have a recessive lethal or sterile phenotype allele conversion (ie conversion from heterozygous to homozygous for the disruption) should occur after the phase when the homozygote is deleterious (eg at meiosis).

If the gene disruption does not have a strong deleterious effect (for example on host survival or reproduction), for example when seeking to transform the population rather than reduce its numbers, then it may not matter if allele conversion is germ-line specific or also occurs in the somatic tissue.

Even in species that are predominantly haploid, with only a brief diploid phase, one could target a gene encoding a protein needed for the entry into meiosis, and then have homing occur during meiosis. Alternatively, some predominantly haploid taxa (including malarial Plasmodium) have an extended post-meiotic syncytial phase, and so in these species one might also target a protein needed during meiosis (eg a synaptonemal complex protein), and have homing occur during the syncytial phase.

The method may comprise the step of preparing a modified organism. Thus, the method may comprise the step of disrupting a selected gene in the germline of an organism which is capable of sexually reproducing with an organism of the target population, by inserting into the selected gene a sequence-specific nonMendelian selfish gene. Methods by which this may be done are discussed further below and in the Examples. The method may further comprise the step of preparing progeny of the prepared modified organism. It will be appreciated that the progeny will also be modified.

Thus, the invention provides a method for genetically modifying a target population of an organism, comprising the steps of
1. preparing a modified organism by disrupting a selected gene in the germline of an organism which is capable of sexually reproducing with an organism of the target population, by inserting into the selected gene a sequence-specific nonMendelian selfish gene.
2. introducing the modified organism (which term includes progeny of the organism prepared as indicated in step 1) into the target population.

It is preferred that the organism is cellular, though it may alternatively be a virus. It is further preferred that the organism is a sexually reproducing eukaryote, preferably multicellular. In particular, it is preferred that the organism is a plant, insect, mollusc, arachnid, amphibian, reptile, rodent or other mammal. It is strongly preferred that the organism is not a human. In relation to certain embodiments of the invention, for example in which a population may be eradicated as a consequence of the genetic modification, that the organism is a pest, for example in relation to agricultural (including forestry) or domestic plants or animals, or to humans.

It is preferred that individuals of the target population (including the introduced modified individuals) are able to reproduce freely and randomly ie without selective human intervention in relation to choice of mate or survival of progeny. Thus, it is preferred that the target population is a sexually-reproducing population, in which the modified organism is allowed to sexually reproduce with the non-modified organisms in the population.

It is therefore preferred that the target population is not a controlled population such as a crop in which reproduction is limited or prevented, for example by harvesting of the seed for food, or a livestock population in which reproduction is controlled by man.

The methods of the invention may be used to knock-out a gene (which term includes a cis acting-control region) in a substantial fraction of the population. Thus, the methods may be used to transform the population (ie alter its phenotype, for example in relation to pest or parasite resistance), but leave numbers more-or-less intact. Alternatively, the methods may be used to impose a load on the population. This may be useful in reducing densities or eliminating the population/species, for example by targeting genes whose knock-outs are harmful (e.g., lethal or sterile) when homozygous (effects may or may not be maternal or conditional), or by increasing the efficacy of sterile male releases. Alternatively, the methods may be used to create conditions for the spread of a resistant gene which is somehow different from what was originally there (for example being linked to a gene of interest, or having some amino acid difference). The methods may also be used to alter the sex ratio of the population. For example, by creating double-strand breaks in the X-chromosome, or by disrupting a cis-acting region necessary for meiotic segregation of the X-chromosome, sperm can be made disproportionately Y-bearing, leading to an increase in the frequency of males.

These uses of the methods are discussed in further detail in the Examples.

Depending on the population size, the properties of the nonMendelian selfish gene and the effect of the gene disruption, it is preferred that more than a single modified organism is introduced into the population. It is preferred that the number of modified organisms introduced into the population is sufficient for the gene disruption to spread through at least 50, 60, 70, 80, 90 or 95% the population after about 10 to 100 (preferably between 20 and 80 or 30 and 70) generations, or for at least 50, 60, 70, 80 90 or 95% of the initial population to be eradicated (or predicted to be eradicated) after about 10 to 100 (preferably between 20 and 80 or 30 and 70) generations. This is discussed further in the Examples. Generations may be assessed directly by observation or calculated from estimated generation times, as will be well known to those skilled in the art.

Thus, the method may in addition to a step of preparing or providing a modified organism, comprise the step of generating progeny of the modified organism and introducing such progeny into the target population.

In a preferred embodiment the sequence-specific nonMendelian selfish gene is a homing endonuclease gene (HEG), preferably a recombinant HEG that does not exist in nature, for example encoding a recombinant endonuclease that does not exist in nature. As noted above, a HEG comprises a polynucleotide sequence necessary for an endonuclease to be expressed when the HEG is inserted at the site in the host organism genome where the expressed endonuclease cleaves. Preferably, the HEG comprises a polynucleotide sequence encoding the endonuclease and any additional sequences required for the endonuclease to be expressed, for example a promoter. A recombinant endonuclease with a desired sequence specificity (and polynucleotide encoding it) may be prepared as described in the Examples, for example by fusing a DNA binding portion with the desired sequence specificity (for example a zinc-finger DNA binding domain) with a non-specific DNA cleavage domain, for example from a type ES restriction endonuclease. Alternatively, the recombinant endonuclease may be a modified naturally occurring HEG (of which there are 3-4 classes, as discussed above and in the Examples).

Thus, it is preferred if the endonuclease is a hybrid polypeptide which do not occur in nature. For example, it is preferred if the nucleic acid binding portion is derived from one protein and that the nuclease or cleavage portion is derived from a different protein and that the molecular configuration does not arise in nature, for example through chromosome translocation events. The proteins from which the nucleic acid binding portion and the nuclease portion are derived may be from the same species or from different species. For example, the nucleic acid binding portion may be a DNA binding portion of nuclear receptor binding protein (for example a plant or insect steroid receptor protein) and the cleavage portion may be from the restriction endonuclease FokI from Flavobacterium okeanokoites.

Thus, in a particular preferred embodiment the polypeptide of the invention is one which is produced by genetic engineering means wherein the nucleic acid binding portion and the cleavage portion are selected as is described in more detail below.

The DNA binding portion and the cleavage portion are fused such that the fusion polypeptide may be encoded by a nucleic acid molecule. Suitably, the DNA binding portion and the cleavage portion are joined so that both portions retain their respective activities such that the polypeptide may bind to a site present in the organism's genome and, upon binding, the cleavage portion is still able to cleave the desired target nucleic acid sequence. The two portions may be joined directly, but they may be joined by a linker peptide. Suitable linker peptides are those that typically adopt a random coil conformation, for example the polypeptide may contain alanine or proline or a mixture of alanine plus proline residues. Preferably, the linker contains between 10 and 100 amino acid residues, more preferably between 10 and 50 and still more preferably between 10 and 20. In any event, whether or not there is a linker between the portions of the polypeptide the polypeptide is able to bind its target DNA and is able to cleave DNA thereby permitting gene conversion.

Polynucleotides which encode suitable nucleic acid binding portions, particularly DNA binding portions are known in the art or can be readily designed from known sequences such as from known sequences contained in scientific publications or contained in nucleotide sequence databases such as the GenBank, EMBL and dbEST databases. References describing methods by which zinc finger polypeptides with desired sequence binding specificity may be designed or selected are mentioned in the Examples.

Polynucleotides which encode suitable linker peptides can readily be designed from linker peptide sequences and made.

Thus, HEGs of the invention can readily be constructed using well known genetic engineering techniques.

A variety of methods have been developed to operably link polynucleotides, especially DNA, to other polynucleotides, including vectors, for example via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerising activities.

The combination of these activities therefore generates blunt-ended DNA segments. The blunt-ended segments are then incubated with a large molar excess of linker molecules in the presence of an enzyme that is able to catalyse the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

A desirable way to modify the DNA encoding the HEG of the invention is to use the polymerase chain reaction as disclosed by Saiki et al (1988) Science 239, 487-491. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

Methods of joining a polynucleotide to a nucleic acid vector are, of course, applicable to joining any polynucleotides.

The DNA (or in the case of retroviral vectors, RNA) may then be expressed in a suitable host to produce the recombinant endonuclease. Thus, the DNA encoding the recombinant endonuclease may be used in accordance with known techniques, appropriately modified in view of the teachings contained herein, to construct an expression vector, which is then used to transform an appropriate host cell for the expression and production of the recombinant endonuclease. Such techniques include those disclosed in U.S. Pat. Nos. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

The DNA (or in the case of retroviral vectors, RNA) encoding the HEG may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA are then cultured for a sufficient time and under appropriate conditions known to those skilled in the art in view of the teachings disclosed herein to permit the expression of the endonuclease, which can then be recovered if desired. Alternatively, expression of the endonuclease may be useful in introducing the HEG to the correct site in the target organism's nucleic acid.

A recently published technique for Drosophila for targeting an insert to a particular location in the genome, which may be applicable to other species, is that of Rong & Golic (2000) Science 288:2013-8.

Expression and transformation systems are known for many organisms, including bacteria (for example E. coli and Bacillus subtilis), yeasts (for example Saccharomyces cerevisiae), filamentous fungi (for example Aspergillus), plant cells, animal cells and insect cells.

The vectors include a prokaryotic replicon, such as the ColE1 ori, for propagation in a prokaryote, even if the vector is to be used for introduction into and/or expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention. It is preferred that the promoter is one which can be regulated. It is particularly preferred if the promoter is an inducible promoter which can be selectively induced at an appropriate time once the vector has been introduced into the eukaryotic cell. It will be appreciated that upon induction, the polypeptide of the invention may be expressed in the cell and exert its effect. In this situation, induction of expression of the polypeptide of the invention leads to suppression of the targeted gene. Inducible promoters are known in the art for many eukaryotic cells including plant and animal cells. These include heat-shock-, glucocolticoid-, oestradiol-, and metal-inducible promoter systems.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Plant transformation vectors are well known in the art. For example, vectors for Agrobacterium-mediated transformation are available from the Centre for the Application of Molecular Biology to International Agriculture, GPO Box 3200, Canberra, ACT 2601, Australia (cambia@cambia.org.au).

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA. With regard to plant cells and whole plants three plant transformation approaches are typically used (J. Draper and R. Scott in D. Grierson (ed.), “Plant Genetic Engineering”, Blackie, Glasgow and London, 1991, vol. 1, pp 38-81):

i) Agrobacterium-mediated transformation, using the Ti plasmid of A. tumefaciens and the Ri plasmid of A. rhizogenes (P. Armitage, R. Walden and J. Draper in J. Draper, R. Scott, P. Armitage and R. Walden (eds.), “Plant Genetic Transformation and Expression—A Laboratory Manual”, Blackwell Scientific Publications, Oxford, 1988, pp 1-67; R. J. Draper, R. Scott and J. Hamill ibid., pp 69-160);

Agrobacterium-mediated transformation is also described in Hooykaas & Schilperoot (1992) Plant Mol. Biol. 19, 15-38; Zupan & Zambryski (1995) Plant Physiol. 107, 1041-1047; and Baron & Zambryski (1996) Curr. Biol. 6, 1567-1569.

ii) DNA-mediated gene transfer, by polyethylene glycol-stimulated DNA uptake into protoplasts, by electroporation, or by microinjection of protoplasts or plant cells (J. Draper, R. Scott, A. Kumar and G. Dury, ibid., pp 161-198). Direct gene transfer into protoplasts is also described in Neuhaus & Spangenberg (1990) Physiol. Plant 79, 213-217; Gad et al (1990) Physiol. Plant 79, 177-183; and Mathur & Koncz (1998) Method Mol. Biol. 82, 267-276;

iii) transformation using particle bombardment (D. McCabe and P. Christou, Plant Cell Tiss. Org. Cult., 3, 227-236 (1993); P. Christou, Plant J., 3, 275-281 (1992)).

Some species are amenable to direct transformation, avoiding a requirement for tissue or cell culture (Bechtold et al (1993) Life Sciences, C.R. Acad. Sci. Paris 316, 1194-1199).

Agrobacterium-mediated transformation is generally less effective for monocotyledonous plants for which approaches ii) and iii) are therefore preferred. However, Agrobacterium is capable of transferring DNA to some monocotyledenous plants if tissues containing “competent” cells are infected (see Hiei et al (1997) Plant Mol. Biol. 35, 205-218). In all approaches a suitable selection marker, such as kanamycin- or herbicide-resistance, is preferred or alternatively a screenable marker (“reporter”) gene, such as β-glucuronidase or luciferase (see J. Draper and R. Scott in D. Grierson (ed.), “Plant Genetic Engineering”, Blackie, Glasgow and London, 1991, vol. 1 pp 38-81).

Electroporation is also useful for transforming and/or transfecting cells and is well known in the art for transforming yeast cell, bacterial cells, insect cells, vertebrate cells and some plant cells (eg barley cells, see Lazzeri (1995) Methods Mol. Biol. 49, 95-106).

For example, many bacterial species may be transformed by the methods described in Luchansky et al (1988) Mol. Microbiol. 2, 637-646 incorporated herein by reference. The greatest number of transformants is consistently recovered following electroporation of the DNA-cell mixture suspended in 2.5×PEB using 6250V per cm at 25 μFD.

Methods for transformation of yeast by electroporation are disclosed in Becker & Guarente (1990) Methods Enzymol. 194, 182.

In insects, genetic engineering may be done by injection into the eggs. This is the standard technique for Drosophila, which has recently also been used for Anopheles (see Catteruccia et al (2000) Nature 405, 959-961) & Aedes. Cattericcia et al (2000) also includes references describing transformation techniques that may be used with other insect pests, for example the Mediterranean fruit fly Ceratitis captata, the yellow fever mosquito Aedesaegypti and the flour beetle Tribolium castaneum.

Methods for transforming nematodes, for example Caenorhabditis elegans are described in, for example Sulston & Hodgkin (1988) Methods in The Nematode, Caenorhabditis elegans Ed Wood, W. Cold Spring Harbor Press, Plainview, N.Y., pp 587-606 and on the website http://elegans.swmed.edu/.

Successfully transformed cells, ie cells that contain a sequence-specific nonMendelian selfish gene, for example a HEG, can be identified by well known techniques. For example, cells resulting from the introduction of an expression construct encoding the endonuclease can be grown to produce the endonuclease. Cells can be harvested and lysed and their DNA content examined for the presence of the DNA using a method such as that described by Southern (1975) J. Mol. Biol. 98, 503 or Berent et al (1985) Biotech. 3, 208. Alternatively, the presence of the protein in the supernatant can be detected using antibodies as described below.

In addition to directly assaying for the presence of recombinant DNA, successful transformation can be confirmed by well known immunological methods when the recombinant DNA is capable of directing the expression of the polypeptide. For example, cells successfully transformed with an expression vector produce polypeptides displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium.

In relation to plants, it is envisaged that the invention includes single cell derived cell suspension cultures, isolated protoplasts or stable transformed plants. In the latter case it may be preferred if the endonuclease (or any accompanying “foreign” gene) is expressed using an inducible promoter system to avoid potentially lethal effects of gene down-regulation during regeneration of homozygous plants.

Although the sequence-specific nonMendelian selfish gene may be introduced into any suitable host cell, it will be appreciated that they are primarily designed to be effective in a selected organism, for example in appropriate animal or plant cells, particularly those that have one or more sites within their DNA to which the endonuclease may bind and cleave.

It will be readily appreciated that introduction of the sequence-specific nonMendelian selfish gene, particularly a HEG into an animal or plant cell, will allow targeting of, for example, the expressed endonuclease to an appropriate binding site within the DNA (and which is bound by the DNA-binding portion of the polypeptide) and allow for the nucleic acid at or associated with the target binding site to be cleaved so as to lead to introduction of the HEG at the cleavage site. Typically, the selfish gene is selected so that it targets a selected gene. Thus, suitably, the targeted gene has a site which is bound by the DNA binding portion of, for example, the endonuclease associated with it. The site which is so bound may be within the gene itself, for example within an intron or within an exon of the gene; or it may be in a region 5′ of the transcribed portion of the gene, for example within or adjacent to a promoter or enhancer region; or it may be in a region 3′ of the transcribed portion of the gene.

In other embodiments the sequence-specific nonMendelian selfish gene may be a retrohoming group II intron or a site-specific LINE-like transposable element, preferably a recombinant retrohoming group II intron or a recombinant site-specific LINE-like transposable element, as discussed further in the Examples.

These elements get transcribed into RNA, which codes for a reverse transcriptase, which then reverse transcribes the RNA into DNA, and inserts it into a sequence-specific place in the genome. Guo et al. 2000 Science 289:452-457 show how to engineer a group II intron to insert into a specific sequence. Yang et al. 1999 PNAS 96:7847-7852 suggest that the sequence specificity of LINEs is given by zinc-finger and c-myb-like DNA binding motifs in the protein. These sites may be engineered to change the sequence specificity.

Retrohoming group II introns are in many ways similar to homing endonuclease genes (HEGs): they are optional genetic elements with no known host function that target a particular locus and, by virtue of their catalytic activities, contrive to increase in frequency at that locus, without being of any (known) selective benefit to the host.

This mechanism has been best studied for the aI1 and aI2 introns of the yeast Saccharomyces cerevisiae and for the L1.LtrB intron of the bacterium, Lactococcus lactis (Eskes et al. (1997) Cell 88:865-874; Cousineau et al. (1998) Cell 94:451-462; Guo et al. (2000) Science 289:452-457). In each case the intron is contained within a host gene (cox1, a mitochondrial gene, for the two yeast introns), and encodes its own multi-functional protein which helps its spread. In cells with both intron⁺ and intron⁻ copies of the host gene (recall that yeast mitochondria are biparentally inherited), this intron-encoded protein (IEP) helps in splicing the intron out of the RNA transcript of the intron⁺ host gene (i.e., it acts as a ‘maturase’). The intron and protein remain associated, and together they recognize the intron⁻ copy of the host gene and reverse-splice the intron into the sense strand of DNA. The protein then nicks the anti-sense strand downstream of the insertion site, and uses the resulting 3′ end of DNA as a primer to reverse transcribe the intron into DNA. Final ligation of the cDNA copy of the intron to the flanking host gene is then done by host repair mechanisms.

As for HEGs, the recognition sequence used by group II introns is long: for is example, for aI2 it is 31 bp, running from 21 bp upstream of the insertion site to 10 bp downstream (Guo et al. 1997 EMBO J16:6835-48). Both the intron and the IEP are involved in recognizing the target site: positions −12 to +1 are recognized primarily by base-pairing between the target DNA and the intron RNA (positions numbered from the insertion site), while sequences flanking this are recognized primarily by the protein. Protein recognition of the upstream sequence leads to DNA unwinding, allowing the intron to base-pair with the DNA, while protein recognition of the downstream sequence occurs after reverse splicing, and allows the protein to nick the DNA and begin reverse transcription. The recognition process for aI1 is very similar (ang et al. (1998) J. Mol. Biol. 282:505-523). For L1.LtrB, the recognition sequence is 35 bp, running from positions −26 to +9, with positions −13 to +1 recognized primarily by the intron, and flanking positions by the protein (Mohr et al. (2000) Genes & Development 14:559-573; Guo et al. (2000) Science 289:452-457). As for HEGs, not all positions within these recognition sequences are equally important. The fact that recognition depends in part on RNA-DNA base pairing means that it is relatively easy to engineer group II introns with novel recognition sequences (Guo et al. (2000) Science 289:452-457).

For the yeast elements, this “retro-homing” pathway (so named because it involves reverse transcription of RNA into DNA) can be complemented by purely DNA-based homing: the intron and IEP can together cause a double strand break in the target site which can be repaired using the intron⁺ gene as a template. In two different crosses, the frequency of this alternative DNA-level pathway varied from about 10-40% of homing events (Eskes et al. (1997) Cell 88:865-874).

The consequence of all this activity is rates of inheritance that are similar to those achievable with HEGs.

LINEs (Long Interspersed Nuclear Elements; also known as non-LTR or poly(A) retrotransposons) are a widespread class of transposable elements which can be found in the genomes of most eukaryotes. They show the full range of copy number: I elements of Drosophila melanogaster, for example, are present in about 20 copies per genome, whereas there are about 20,000 full length copies of L1Md in the mouse genome, and another 150,000 partial copies, constituting about 10% of the genome. In the plant Lilium speciosum there are about 250,000 copies of Del2 (ca. 4% of the genome—Leeton & Smyth (1993) Mol. Gen. Genet. 237:97-104). Curiously, there are no LINEs to be found in the yeast Saccharomyces cerevisiae, though they do exist in other fungi (Kemplen & Kuck (1998) Mol. Gen. Genet. 237:97-104). Like DNA transposons, most LINEs can integrate at many different places in the genome, but there does exist a substantial minority which are site-specific, and insert themselves only into particular places in multi-copy host genes. For example, R1 is a LINE in D. melanogaster which inserts specifically at a particular nucleotide position in the 28S rRNA genes; R2 is another LINE which inserts at another position 74bp upstream of R1 (Jakubczak et al. (1990) J. Mol. Biol. 212:37-52). About half of the 28S rRNA genes have one or the other insert, and consequently are non-functional. These elements are widespread among insects, having been described from the rRNA genes of 9 orders of insects (Jakubczak et al. (1991) PNAS 88:3295-3299; see also Burke et al. (1995) NAR 23:4628-34; Xiong & Eickbush (1993) NAR 21:1318). Site-specific LINEs have also been found in the pentanucleotide repeats at the telomeres of Bombyx mori (Takahashi et al. (1997) NAR 25:1578-1584) and in spliced leader RNA (SL RNA) genes of trypanosomes (rev. in Aksoy (1991) Parasitology Today 7:281-285) and nematodes (Malik & Eickbush (2000) Genetics 154:193-203). The site-specific LINEs also include Tx1 elements of Xenopus frogs, and Zepp elements of Chlorella algae that specifically target pre-existing copies of themselves (Higashiyama et al. (1997) EMBO J. 16:3715-3723).

All full length LINEs encode a multi-functional enzyme with domains for DNA binding, DNA cleavage, and reverse transcription of RNA into DNA. Many LINEs also encode a second protein which binds RNA, but its function is not yet clear (Hohjoh & Singer (1997) EMBO J. 16:6034-6043; Dawson et al. (1997) EMBO J. 16: 4448-4455). Retrotransposition is thought to occur by the following steps (Luan et al. (1993) Cell 72:595-605; Boeke (1997) Nat. Genet. 16:6-7):

(1) An element is transcribed into RNA. Unlike most (but not all) host genes which have their promoter(s) upstream of the transcription start site, LINEs have an internal promoter. By carrying its own promoter, the element increases the probability that it will be transcribed regardless of where it happens to be in the genome.

(2) As with most host genes, the RNA is cleaved at the 3′ (downstream) end at a polyadenylation signal, and a poly(A) tail added. There are no introns to be spliced out. The RNA then moves to the cytoplasm.

(3) The RNA is translated to make its one or two proteins. As it is being made, or shortly thereafter, the protein(s) binds to the very RNA molecule from which it is being translated.

(4) After translation, the protein-RNA complex moves back to the nucleus, and the protein binds to and nicks (cuts a single strand of) the host DNA. For most LINEs this can occur at very many places in the genome, but there is a preference for A-T rich DNA, and in particular for the nick to expose a short run of, say, 4 T's, which can bind to the poly(A) tail of the RNA (Feng et al. (1996) Cell 87:905-916; Jurka (1997) PNAS 94:1872-1877). For the site-specific LINEs, the protein has a particular recognition sequences and only nicks the DNA there.

(5) The exposed 3′ end of host DNA is used to ‘prime’ reverse transcription of the RNA into DNA. (Like normal host DNA polymerases, reverse transcriptase cannot synthesize a complementary strand de novo, but must instead have something pre-existing which it then extends.) Reverse transcription starts at the poly(A) tail, and works up to the beginning of the transcript. Very often it does not get all the way to the front, and a 5′ truncated element (with the front part missing) ends up being inserted.

(6) After reverse transcription (making a hybrid RNA-DNA molecule with the DNA strand attached at one end to the host genome), the other strand of host DNA is cut and somehow the second strand of the LINE element is synthesized, replacing the RNA with DNA, and all loose ends get ligated to the host genome to create an integrated LINE element.

Several important differences distinguish LINE transposition from that of DNA transposons. First, mutations that occur during the copying process happen to the element at the new site, not the one at the ancestral site. In a public lecture, M. Singer compared LINEs to faxes, in which the original stays at home and a potentially degraded copy goes elsewhere. Second, excision is no part of the transposition process, which means that once acquired, LINEs are unlikely to be lost from a site. Thus, reversions are likely to be much less frequent. Finally, and most importantly, the cis-activity of LINEs—the fact that the reverse transcriptase protein predominantly uses as template the very same RNA molecule from which it was translated—is very significant. It means that defective elements cannot accumulate in the genome in the same way that they can for DNA transposons. Defective elements are created with great frequency, due to truncations and point mutations (transcription and reverse transcription being significantly more error-prone than DNA replication), but once created they are much less likely to replicate again than functional elements (for evidence of weak trans-complementation, see Pelisson et al. (1991) PNAS 88:4907-4910; Jensen et al. (1994) NAR 22:1484-1488; Busseau et al. (1998) Genetics 148:267-275). Of the 100,000 copies of L1 in the human genome, about 3000-4000 of which full length, only 30-60 are thought to be capable of retrotransposition (Sassaman et al. (1997) Nat. Genet. 16:37-43). This is not so different from, say, the number of active I elements in D. melanogaster. About 3000 active L1's are thought to exist in the mouse genome (DeBerardinis et al. (1998) Nat Genet. 20:288, as cited in Moran et al. 1999).

Phylogenetic analysis indicates that throughout transposable element evolution there has been a strong theme of domains being gained and lost, with new acquisitions coming both from other transposable elements and from host genes. Among the best analysed in this regard are the LINEs (Malik et al. (1999) Mol. Biol Evol. 16:793-805; Yang et al. (1999) PNAS 96:7847-7852). All LINEs, of course, share a reverse transcription (RT) domain, and if this is used to construct phylogenies, than the ancestral LINE appears to have encoded a single ORF and been site-specific, with a restriction-enzyme-like endonuclease (REL-endo) domain downsteam of the RT domain (FIG. 7 from Malik et al. 1999). In some lineages, this REL-endo domain was replaced by an AP endonuclease (APE) domain acquired from the DNA repair machinery of the host cell. (Some of those with the APE domain are site-specific and some not; it is not yet clear whether site-specificity is ancestral or derived (or both) among these elements.) Among those elements with an APE domain, it appears that one lineage (including L1 from humans) has gained a second ORF which has a leucine zipper motif, and another lineage has gained a second ORF with gag-like cysteine-histidine zinc finger motifs (similar to the gag protein of LTR retroelements). These are both nucleic acid binding motifs. These cysteine-histidine motifs are also found sporadically in the original ORF both upstream and downstream of RT. Finally, among those with a gag-like ORF, RNase H domains appear sporadically on the RT phylogeny, and was apparently gained at least once from the host cell, and then lost several times. This domain is thought to eliminate the RNA template after reverse transcription; for elements lacking it, this function is presumably carried out by host RNase H activity.

The sequence-specific nonMendelian selfish gene may be targeted to a sequence which occurs naturally, or in a recombinant sequence present in the target organism; for example, the target sequence may be present in a recombinant sequence present in a genetically modified organism. However, in most circumstances it is preferred that the target sequence is naturally occuring.

The target sequence may be between about 15 and 30 nucleotides long, preferably between about 20 and 30 nucleotides long. It is preferred that the target sequence occurs a limited, known number of times, in most cases preferably only once in the genome of the target organism. This may be determined using techniques well known to those skilled in the art, for example Southern blotting or using computer-based sequence and database searching/comparisons.

In preferred embodiments, the sequence-specific nonMendelian selfish gene is germ-line-specific. By this is meant that spread of the selfish gene does not take place in somatic tissue. In somatic tissue, an organism that is heterozygous for the nonMendelian selfish gene remains heterozygous, and it will be able to live even if the gene is lethal when homozygous. In the organism's germ-line, the selfish gene is able to spread, so that nonMendelian transmission of the selfish gene occurs.

Germ-line specificity required when knocking out a recessive lethal or sterile gene. Knocking out a gene with little selective effect (for example when attempting to transform a population rather than eradicate it) does not require germ-line specificity. If the target gene is only expressed in larvae, or in somatic tissues, then the promoter may be adult-specific, or germline-specific, respectively.

When the selfish gene is a HEG, for example, expression of the endonuclease encoded by the HEG is preferably under the control of a germ-line specific (or meiosis-specific) promoter. This means that the endonuclease is not expressed in somatic tissue. In germ-line tissue, the endonuclease is expressed, and cuts wild-type host gene (that does not contain the endonuclease); the gene containing the endonuclease will not be cut because the presence of the HEG interrupts the recognition site. The cut gene is repaired by copying the uncut gene containing the endonuclease, thereby converting both alleles to the endonuclease-containing form.

The disrupted host gene may be a recessive lethal or sterile. By “recessive” is meant that there is negligible difference between the wild-type phenotype and the phenotype of a heterozygote. Population engineering using the sequence-specific nonMendelian selfish gene to disrupt a gene is considered to be evolutionarily stable in the face of new mutations: mutations in the nonMendelian element will simply lose their nonMendelian inheritance, and be lost from the population. In addition, gene disruption (i) avoids the difficulty of knowing which gene to introduce—most species are considered to have at least 10's or 100's of recessive female-specific sterility genes which could be targetted; (ii) is reversible, in the sense of being able to introduce a resistant gene into a population, which will lead to the extinction of the selfish gene. Further, it is considered to be more efficient than other methods, in that a greater load is imposed per element introduced. These features are discussed further in the Examples.

In further embodiments, when a single copy of the disrupted gene has a substantial (or non-negligible) effect on fitness, (for example when the phenotype of the heterozygote differs from that of the wild-type, but not by as much as the phenotype of the homozygote for the disrupted gene differs from that of the wild-type), this allows one to eradicate a local population, but rare emmigrants would not be able to infect and eradicate other populations. Depending on the degree of detrimental effect caused by a single copy of the disrupted gene, an initial introduction of, for example, 0.1% of the population with a disrupted gene would not lead to spread of the disrupted gene through the population, but initial introduction of, for example, 10%, 20% or 50% of the population with a disrupted gene may lead to spread of the disrupted gene throughout the population and the population's extermination. If this was the case, then a big release in the target population would eradicate it, but if some of the target population were to escape to a second population, then the disrupted gene would not spread through that population. This would be useful in controlling a population in a particular region without harming neighbouring populations of the same organism.

In a further embodiment, targetting synthetic lethal or sterile genes (in which two or more genes have to be disrupted to get the phenotype) would also help in containing the disrupted gene to the population of interest, preventing unwanted escapees into other populations.

As noted above, in a preferred embodiment, the sequence-specific nonMendelian gene is a homing endonuclease gene (HEG), which may be engineered to cleave at a particular selected target sequence. The HEG may be used to disrupt target genes in a given genome and drive this disruption through the population. The endonuclease gene would be inserted in that recognition sequence. When the gene is transcribed, the endonuclease would cut the recognition sequence of the other copy of the gene. That break would then be repaired using the engineered gene, thus making the cell homozygous for the interruption. If this endonuclease is put under the control of a meiotic promotor, the somatic genotype will be heterozygous, but upon meiosis, all haploid cells will carry the disrupted gene and consequently pass it on to all its offspring. As the spread of these selfish genes depends on how they influence the fitness of an individual, the interrupted gene preferably has a zero or negligible detrimental effect in the heterozygous state, thus interruption of a recessive gene is desirable. The spread of such a gene is modelled in the Examples.

When disrupting a gene in order to eradicate a population, it is preferred the target gene is a recessive sterile, still more preferably a recessive substerile, if the substerility is correct.

Alternatively, the target gene may be a recessive lethal. Thus, once the gene has sufficiently penetrated the population, somatic homozygotes will appear and die. Modelling shows that targeting a recessive lethal gene allows the disrupted gene to reach a higher frequency in the population than targeting a dominant lethal gene. The more frequent the disrupted gene (comprising the nonMendelian selfish gene) is, the stronger the subsequent drive through the population is.

The population may be a population of a pest; for example it may be a population in a confined space such as a lake or greenhouse or on an island. It is preferred that the population is not a laboratory population. The method may be most useful when dealing with organisms that have a short generation time in relation to the period of time over which it is desired to reduce, eliminate or alter the population.

For example, the technique may be useful in eradicating or controlling a population of animals with a generation span of a few years (for example rodents or goats) over a period of 20 to 50 years. Such animals may be unwanted colonising species which are detrimental to a previously-established ecosystem.

The method may be used in altering the balance of insects or microorganisms, for example those associated with food crops or livestock.

The method may also be used to interrupt other, non-lethal genes, e.g. a gene that confers a pesticide resistance onto a crop, thus making the pest susceptible to the pesticide again. For example, insects, nematodes or fungi may be rendered susceptible to appropriate pesticides.

Introducing two or more disruptions in independently inherited recessive lethal genes may speed eradication.

The modified organism may further comprise an allele of the selected gene which the sequence-specific nonMendelian selfish gene (for example a HEG) is not able to insert into (resistant allele). This may be useful in replacing in the population the allele into which the selfish gene is able to insert with a different allele (into which the selfish gene cannot insert), which may have different properties. Thus, the resistant allele may have a different amino acid sequence, or may have a foreign gene linked to it (as discussed further below), or may be embedded in a chromosomal rearrangement.

Alternatively, the resistant allele may be present in a second modified organism which may also be introduced into the target population. This may be done before, after or at the same time as the first modified organism, preferably at the same time or after the first modified organism.

The effect of gene disruption on a population may also be reversed by introducing organisms which have a resistant allele, for example which have their nucleic acid sequence at the recognition site altered (masked by virtue of redundancy of genetic code) without affecting the aa code, so that the homing endonuclease is not able to cleave the replacement gene. It is considered that the cleavage-resistant gene would spread rapidly through a population due to natural selection, particularly a population with a high frequency of the disrupted gene.

The endonuclease (or site-specific nonMendelian gene) may also have other properties. For example, it may be a physiologically active protein which may have harmful or beneficial effects.

A further aspect of the invention provides a method for genetically modifying a target population of an organism, comprising the steps of
1. Introducing a homing endonuclease gene (HEG) into the germline of an organism which is capable of sexually reproducing with organisms of the target population;
2. introducing the modified organism into the target population.

As noted above, the HEG may be used to disrupt a selected gene in the organism. Alternatively, the HEG may be introduced together with a gene that it is desired to introduce (“foreign” gene) into the target population. The gene is intended to confer (either when heterozygous or homozygous) on the organism a desired property, for example pesticide susceptibility or resistance to a parasite, for example the malarial parasite. As discussed further in the Examples, the HEG and foreign gene may be introduced into the cell as a single construct and insert at the same site in the host genome. Alternatively, there may be more than one cleavage site for the HEG in the host genome, and the gene to be introduced and HEG may be inserted into different cleavages sites. Thus, the HEG and gene to be introduced need not be presented to the cell in a single construct.

As discussed in the Examples, the gene to be introduced may linked in the same construct to an allele resistant to the HEG. This is introduced into the organism together with the HEG.

A further aspect of the invention provides a method for genetically modifying a target population of an organism, comprising the steps of
1. Introducing into the germline of an organism which is capable of sexually reproducing with organisms of the target population a group II intron or site-specific LINE and a gene that it is desired to introduce into the target population (foreign gene);
2. introducing the modified organism into the target population.

It is preferred that the group II intron or site-specific LINE and foreign gene are introduced into the cell as a single construct and insert at the same site in the host genome.

Preferences in relation to genes which it may be desirable to disrupt or introduce, for example in particular circumstances, are indicated in the claims and discussed in the Examples.

Appropriate preferences indicated in relation to the first aspect of the invention, for example in relation to the target population, also apply in relation to the present aspect of the invention.

A further aspect of the invention provides a method for altering the sex ratio in a population of an organism, comprising the steps of
1. Providing a modified organism, wherein the modified organism is capable of sexually reproducing with an organism of the target population, and wherein the modified organism comprises a recombinant polynucleotide encoding and capable of expressing a sequence-specific endonuclease which is capable of cleaving a sequence on a sex chromosome;
2. introducing the modified organism into the target population.

The expression of the endonuclease is preferably under the control of a meiosis-specific (pre-meoitic-specific) promoter. In a preferred embodiment, the endonuclease attacks the sex-chromosome during meiosis in the heterogametic sex.

In some species the sex chromosomes are inactivated prior to meiosis, which may complicate the design of the construct, but this is not so in all species, including many dipterans (McKee & Handel (1993) Chlromosoma 102, 71-80).

In a particularly preferred embodiment, the endonuclease cleaves the sex chromosome at a sequence which is not present on the other sex chromosome present in members of the species. Thus, the endonuclease may cleave the Y chromosome in a species in which males are XY and females are XX at a sequence that is not present on the X chromosome. The Y chromosome is therefore repaired incorrectly or not at all, leading to sperm bearing the broken Y chromosome being non-functional. This biases the sex ratio towards females. Alternatively, if the sequence encoding the endonuclease is on the Y chromosome but the endonuclease attacks a sequence on the X chromosome, then the sperm bearing the (broken) X chromosome are non-functional. Thus, most of the fertilisations will be by Y-bearing sperm, with the result that the population becomes male biased. This may be useful in reducing populations, because population productivity is mostly dependent on female productivity. There is further benefit in relation to mosquitoes, because only the female mosquitoes are vectors for malaria.

It may be particularly beneficial to introduce sequences encoding multiple endonucleases (preferably all on the same chromosome) which attack multiple sites on a sex chromosome. For example, sequences encoding multiple endonucleases which attack the X chromosome may be introduced on the Y chromosome. The sequences encoding the multiple endonucleases will not get separated by recombination and it is highly unlikely that a multiply-resistant X chromosome will appear.

Further embodiments of methods by which the sex ratio may be altered by introducing an endonuclease-coding sequence are described in the Examples.

Preferences for the sequence-specific endonuclease are as indicated above in relation to HEGs. The polynucleotide encoding the endonuclease may be a HEG, as discussed further in the Examples. The polynucleotide encoding the endonuclease may be inserted in a sex chromosome (for example in the opposite sex chromosome to that cleaved by the endonuclease) or an autosome, as discussed in the Examples.

A further aspect of the invention provides a method for altering the sex ratio in a population of an organism, comprising the steps of
1. Providing a modified organism, wherein the modified organism is capable of sexually reproducing with an organism of the target population, and wherein the modified organism comprises a sequence-specific nonMendelian gene (for example a site-specific LINE or group II intron or HEG) targeting a gene (including cis-acting control region) affecting the segregation or viability of the sex chromosome;
2. introducing the modified organism into the target population.

For example, the sequence-specific nonMendelian gene (for example a site-specific LINE or group II intron) may be inserted in a sex chromosome. It is preferred that the trageting (for example insertion) of the sequence-specific nonMendelian gene (for example a site-specific LINE or group II intron) alters the segregation or viability of the sex chromosome such that the ratio of viable gametes (ie gametes capable of producing a viable progeny) carrying the different sex chromosomes is altered.

In an embodiment, the sequence-specific non-Mendelian selfish gene (for example group II intron or site-specific LINE) is inserted into a cis-acting region necessary for X chromosome segregation at meiosis such that segregation is disrupted, so the X chromosome may not segregate properly into the gametes.

A further aspect of the invention provides a polynucleotide comprising a polynucleotide sequence encoding a recombinant sequence-specific endonuclease flanked by the recognition site for the said sequence-specific endonuclease ie the coding sequence for the endonuclease is inserted at the point in the recognition sequence at which the endonuclease cleaves. It will be appreciated that the recombinant sequence-specific endonuclease is not a naturally occuring homing endonuclease, for example as reviewed in Jurica & Stoddard (1999) Cell Mol Life Sci 55, 1304-1326. The recombinant endonuclease may have a DNA binding domain that is a zinc-finger, helix-turn-helix or helix-loop-helix DNA binding domain. Design of DNA binding domains able to bind to a particular selected sequence is discussed in, for example, the following papers: Chandrasegaran & Smith (1999) Chimaeric restriction enzymes: what is next. Biol Chem 380, 841-848; Segal et al (1999) Towards controlling gene expression at will: selection and design of zinc finger domains recognizing each of the 5′-GNN-3′ DNA target sequences PNAS 96, 2758-2763; Guo et al (2000) Group II introns designed to insert into therapeutically relevant DNA target sites in human cells Science 289, 452-457; Bibikova et al (2001) Stimulation of homologous recombination through targeted cleavage by chimeric nucleases Mol Cell Biol 21, 289-297; Buchholz & Stewart (2001) Alteration of Cre recombinase site specificity by substrate-linked protein evolution Nat Biotech 19, 1047-1052; Chevalier & Stoddard (2001) Homing endonucleases: strucutral and functional insight into the catalysts of intron/intein mobility Nucl Acids Res 29, 3757-3774; Santoro & Schultz (2002) Directed evolution of the site specificity of Cre recombinase PNAS 99, 4185-4190; Takahashi & Fujiwara (2002) Transplantation of target site specificity by swapping the endonuclease domains of two LINEs EMBO J 21, 408-417. Wilson et al (2001) The use of mRNA display to select high-affinity protein-binding peptides PNAS 98, 3750-3755 describes methods that may be used to select polypeptides with desired binding characteristics.

A further aspect of the invention comprises a host cell comprises a polynucleotide of the invention. A still further aspect of the invention provides a multicellular organism comprising a polynucleotide of the invention.

The polynucleotide, host cell or organism may further comprise a recombinant polynucleotide comprising a polynucleotide sequence comprising a foreign gene as defined above (ie for introduction into an organism), flanked by the recognition site for the said sequence-specific endonuclease. The foreign gene may confer pesticide susceptibility or resistance to a parasite, for example may confer resistance to a malarial parasite on a mosquito.

A homing endonuclease gene may also be useful for preparing transgenic animals or plants. In particular it may be useful in preparing a transgenic animal or plant homozygous for a gene disruption: using a homing endonuclease gene to generate a gene disruption may increase the number of surviving progeny of crosses between heterozygotes that are homozygous for the disruption.

All patent specifications and other documents referred to herein are hereby incorporated by reference.

The invention is now described in more detail by reference to the following, non-limiting, Figures and Examples.

FIG. 1. Model of ‘homing’ via gene conversion with either the double-strand break-repair or sysnthesis-dependent strand annealing pathway (Colaiacovo et al. 1999; Szostak et al. 1983); light boxes represent an HEG. 1: Recipient (HEG⁻) and donor (HEG⁺) alleles. 2: The endonuclease is transcribed and translated from the HEG⁺ allele and recognises and cuts a specific sequence within the HEG⁻ allele. This sequence is split in two, and therefore destroyed, by the insertion of the HEG. 3: Repair of break using the HEG⁺ allele as a template. 4: Resolution of duplexes.

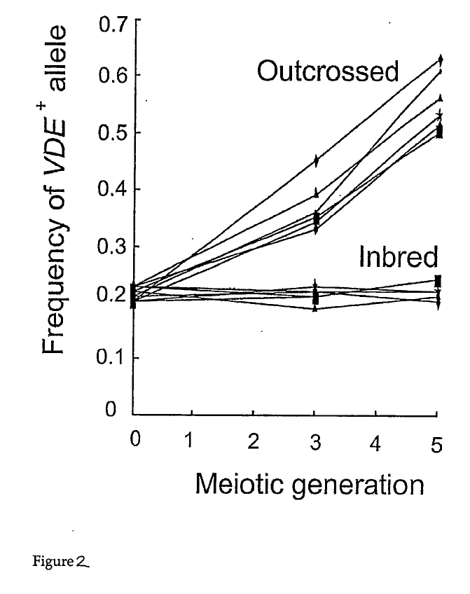

FIG. 2. The change in frequency of VDE within replicate inbred and outcrossed experimental yeast populations. Each symbol represents a replicated inbred and outcrossed population.

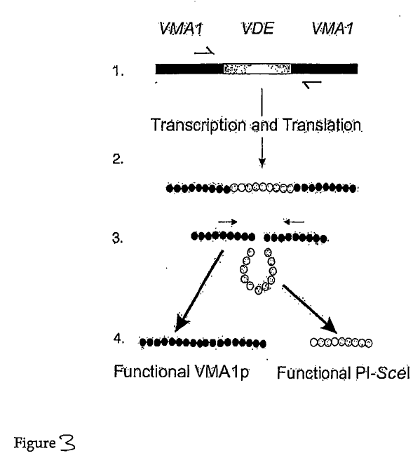

FIG. 3. Representation of protein splicing: dark area—VMA1; light area—VDE1. 1. VMA1 gene with VDE inserted in-frame at the specific site (VDE⁺) at the DNA level. Half arrows indicate primer binding positions. 2. Transcription and translation of entire molecule to rpoduce a protein pre-cursor. 3. The protein product PI-SCEI catalyses its own excision at the protein level, and also ligates the two portions of VMA1p. 4. Production of mature, functional VMA1 and free, functional PI-SceI.

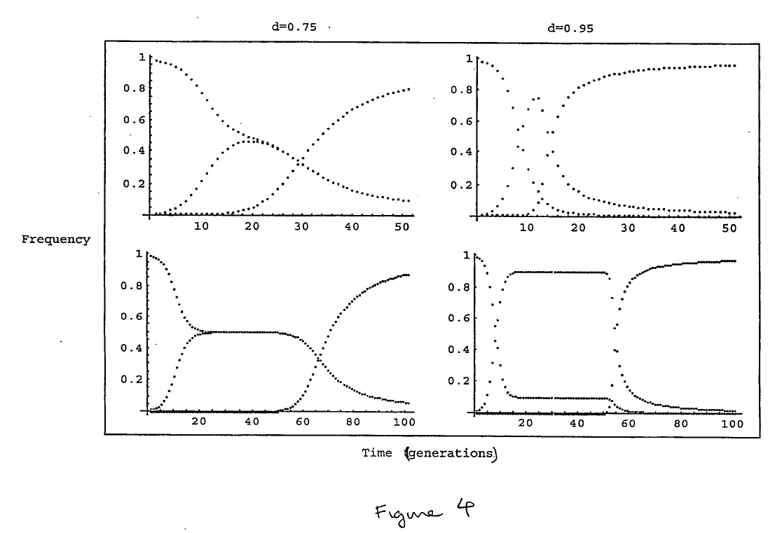

FIG. 4. Effect of release of individuals with a resistant allele.

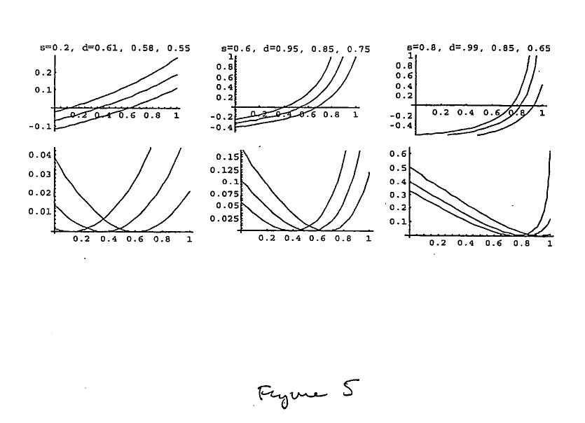

FIG. 5. Selection co-efficient as a function of HEG frequency.

### EXAMPLE 1

### Homing Endonuclease Genes as Tools for Population Genetic Engineering

This example addresses the properties of a gene expressing an engineered site-specific DNA endonuclease and the effect of introducing it into a population.

A site-specific DNA endonuclease with a 25-30 bp recognition sequence that exists only once in the host genome, in the middle of a gene for which knock-out mutations are recessive, may be engineered using techniques for modulating DNA binding specificity well known to those skilled. These techniques include rational design of DNA binding domains and in vitro selection, for example using chip-based binding screens. For example, Chandrasegaran & Smith (1999) Biol Chem 380, 841-848 reviews methods by which endonucleases with a selected target specificity may be prepared. For example, a zinc-finger DNA binding protein may be designed or selected for binding to the desired target sequence; and fused with a sequence-non-specific DNA cleavage domain (for example from a type JIS restriction endonuclease). Design and/or selection of DNA binding domains able to bind to a particular selected sequence is also discussed in, for example, the following papers: Segal et al (1999) PNAS 96, 2758-2763; Guo et al (2000) Science 289, 452-457; Bibikova et al (2001) Mol Cell Biol 21, 289-297; Buchholz & Stewart (2001) Nat Biotech 19, 1047-1052; Chevalier & Stoddard (2001) Nucl Acids Res 29, 3757-3774; Santoro & Schultz (2002) PNAS 99, 4185-4190; Takahashi & Fujiwara (2002) EMBO J21, 408-417; Wilson et al (2001) PNAS 98, 3750-3755.

Many genes for which knock-out mutations are recessive are likely exist in all organisms. The polynucleotide encoding the engineered endonuclease may be put under the control of a germ-line specific (or meiosis-specific) promoter. This gene (homing endonuclease gene: HEG) is used to engineer host individuals so that they carry this gene inserted in the middle of their own recognition sequence. That is, the HEG disrupts both the host gene (so that it is non-functional) and the recognition site (so that it is not cut).

In heterozygous individuals the situation is as follows. In their somatic tissue, they will remain heterozygous, and so they will be able to live even if the gene is lethal when homozygous. In their germ-line, the endonuclease is expressed, and cuts wild-type host gene (that does not contain the endonuclease); the gene containing the endonuclease will not be cut because the presence of the HEG interrupts the recognition site. The presence of the cut chromosome will turn on the cell's repair system, which will often use the homologous chromosome (containing the HEG) as a template for repair (and if not, the site will just get cut again). The result is that that the germ-line is converted from heterozygous to homozygous.

In the ideal case, the host individual itself is fully fertile, but all of its gametes carry the HEG, rather than the Mendelian 50%. Because of this, the gene will increase in frequency in the population. As it does so, the frequency of homozygous individuals expressing the knock-out phenotype will also increase. The eventual fate of the HEG might be fixation, or it might go to some intermediate equilibrium frequency, depending upon the strength of the drive (extent of deviation from Mendelian inheritance) and on the fitness effects of the knock-out (how much the viability and fertility is affected).

In the simplest case of a homogenous panmictic random-mating population with the HEG getting into a proportion d of both eggs and sperm, d>½, and the 3 genotypes (homozygous without HEG, heterozygous, homozygous with HEG, henceforth −−, −+, ++) having fitnesses 1, w_{y}, and w_{z} in both sexes, then the condition for the gene to spread when rare (ie to be able to invade a population) is that

$$d > \frac{1}{2 {w}_{y}} or {w}_{y} > \frac{1}{2 d}$$

and the condition for it to spread when common (ie fix) is

$$d > 1 - \frac{{w}_{z}}{2 {w}_{y}} or \frac{{w}_{z}}{{w}_{y}} > 2 - 2 d$$

So, if one can manage d=0.95, then the HEG will invade as long as the fitness of the knockout in the heterozygous state is greater than 0.526, and it will fix as long as the ratio of homozygous to heterozygous fitnesses is greater than 0.1. If d=0.75, these criteria are 0.67 and 0.5, respectively.

Crucially, note that the system is evolutionarily stable to the appearance of dysfunctional HEGs by mutation. (i) If a mutant loses the ability to recognise or cut the DNA, then it will be selected against and disappear, as it will be harmful to the host (because it will still disrupt the host gene), but will not have the compensating transmission ratio distortion. (ii) If a mutant loses the germ-line specificity, so it is also active in somatic tissue, then again it will again be selected against and disappear, as it will be more disruptive to the host (zygotes created as heterozygotes will have the fitness of homozygotes), with no increase in transmission ratio distortion. (iii) If a mutant loses the site-specificity, so that it cuts at other sites in the genome, then again it is likely to be selected against and disappear, because multiple cuts in a cell are likely to increase the probability of cell death and so reduce host fertility (note that high P-element activity in the germ line reduces Drosophila fertility, often to the point of sterility, and this is thought to be due to the multiple chromosome breaks caused at excision), with no increase in transmission ratio distortion (cuts at ectopic sites will be repaired by the homologous ectopic site, not by the HEG-containing allele). This stability in the face of mutations is a major advantage of the HEG technique over the alternative of driving genes into host populations.

Many (perhaps most) genes may fit the criteria indicated above. The selection coefficient against the knockout must be greater than both the mutation rate to nonfunctional (about 10⁻⁶ in Drosophila) and the reciprocal of the population size; were this not true, then the gene could not be maintained by selection in the face of mutation pressure. Certainly many genes, when knocked out, have no obvious phenotype.

It is useful in relation to pests to disrupt genes for which the knock-outs are not strongly selected against, but which make/render the organism less troublesome. One example is to knock-out a gene which allows a blood-sucking arthropod (eg mosquito, bug, or mite) to act as a vector for pathogens. Or, one could target a gene which gives a pest resistance to some pesticide—either that it has evolved in response to previous pesticide application; or that it just happens to have naturally (that is, one could create super-susceptible populations, that are susceptible to something that they would not normally be susceptible to); or that has been introduced by genetic modification. One might also be able to create a uniquely susceptible population, that is susceptible to something which is harmless to everything else.

The simulation shown in FIG. 4 demonstrates that if a functional host gene exists that is resistant to the HEG then it will increase rapidly in frequency and drive the HEG extinct. Care must therefore be taken to minimise the likelihood that such sequences exist, or arise before the population is eradicated. The first step would be to use mutagenesis experiments and structural studies to choose target genes, and sites within genes, that seem unlikely to be able to change (at the amino acid level) without seriously compromising function. One would also want to choose sites that show little sequence variation in the target population. One would also want to choose sites that show little sequence variation in the target population. One could plausibly sequence 10³ or 10⁴ alleles, and if one engineered an HEG with some redundancy (as naturally occurring HEGs have), to recognise all sequence variants detected, one could thereby ensure that the initial frequency of resistant alleles was less than 1 in 10³ or 10⁴. Indeed, one might be able to go further, and engineer an HEG which could recognise all sequence variants actually detected, plus, say, all possible single nucleotide variants of the observed sequences. Insertion or deletion mutations in the target site may be particularly difficult for the HEG to recognise, and so one will want to choose regions which are well conserved for length across species, or for which structural information suggests that any length variant is likely to be nonfunctional.

This is not to say that recognition site redundancy should be maximised (or, put another way, that sequence specificity should be minimised). If the endonuclease cleaves nonhomologous sites, then it will reduce fitness even when heterozygous, slowing or preventing its spread. Also, for safety, one will want to be able to release a resistant allele. Finally, it may also be safer if the endonuclease does not recognises the homologous sequence in closely related non-target species, so as to reduce the risk of horizontal transfer. Ideally, one wants to target a site that shows little variation within species, but considerable divergence between species (at least at the nucleotide level).

Imposing a Load

Another possible use of the HEG is to target a selectively important gene precisely because it is important, and disrupting it will impose a load on the population, and either reduce the density of the population or drive it extinct. In this section this approach is considered in some detail, using theoretical models to investigate how much load can be imposed on a population. Not surprisingly, load increases with the strength of the transmission ratio distortion (TRD). It also depends upon the type of gene targeted.

Recessive lethals. Perhaps the most obvious class of gene to target is one which, when knocked-out, is a recessive lethal. There are many such genes (for example, Miklos & Rubin (1996) Cell 86:521-529 estimate there are 3600 such loci in Drosophila melanogaster. If an HEG targetting such a gene is introduced at low frequency, then initially it will appear mostly in the heterozygous state, and show drive but no harmful effects. It will therefore increase in frequency, until reaching some equilibrium at which the harmful effects balance the TRD. If we assume a large random mating population (as is done throughout this section) and TRD occurs equally in males and females, then the equilibrium frequency of the HEG will be
\hat{q}=e
where e is the probability that the wild-type allele in a heterozygote is converted to a knock-out. e=0 for Mendelian inheritance. (e is also equal to 2d-1, where d is the fraction of gametes produced by heterozygotes which carry the HEG, with d=0.5 corresponding to Mendelian inheritance. The extent of TRD can be measured using either e or d; here I use e because it leads to simpler equations.) The load imposed upon the population (i.e., the fraction of the reproductive effort which is rendered unproductive) is then equal to the frequency of homozygotes, \hat{q}², and the mean fitness of the population is 1 minus this, or \overline{ŵ}=1−e². For example, if d=0.75, e=q=0.5, L=0.25, and \overline{ŵ}=0.75. If d=0.95, \overline{ŵ}=0.19. That is, four-fifths of zygotes produced will die, and only one-fifth will survive to reproduce. What effect this will have on population size will depend upon the species concerned; this issue will be discussed later. Finally, this load will arise relatively quickly. For example, if the initial frequency of the HEG is 1%, then the load will be equal to 90% of the equilibrium value after only 17 generations for d=0.75, and 12 generations for d=0.95. If one can only manage to release an initial frequency of 0.01%, then it will take 19 generations with d=0.95 (e=0.9).

Some residual viability in the homozygotes can actually increase the load imposed on the population. For example, for d=0.75, the mean fitness decreases to 0.5 as the homozygous fitness increases to 0.5; for d=0.95, mean fitness decreases to 0.1 as homozygous fitness increases to 0.1. Load is highest just at the point at which the HEG can go to fixation. The results are also robust to a certain level of heterozygote impairment (ie the knock-out is incompletely recessive). For example, if the homozygote is lethal and the heterozygote has fitness 90% of the wildtype, then \overline{ŵ}=0.192 (instead of 0.19) and t_{1,90}=14 generations (instead of 12)

Dominant effects. These numbers compare favourably to the case when the effects of the knock-out are dominant. If the knock-out is a dominant lethal, the gene will go extinct in 1 generation. If it has sublethal effects, then it can spread; the condition for the HEG to spread when introduced at a low frequency is
2wd>1
where the fitnesses of the 3 genotypes (−−, −+, ++) are 1, w, and w and d is the proportion of gametes produced by heterozygotes that contain the HEG (d=0.5 being Mendelian inheritance). If this condition is met, then not only will the HEG invade from rare, but it will continue to increase in frequency all the way to fixation. Then, all individuals will have fitness w. For example, if d=0.75, then the most one could reduce fitness would be to 0.67 (marginally better than the recessive lethal case); if d=0.95, the minimum mean fitness would be 0.53 (substantially worse than the recessive lethal case). And, these effect would take time: 190 generations for 90% load for d=0.75, w=0.67; 33 generations for d=0.75, w=0.75; 133 generations for d=0.95, w=0.53. In addition, finding target loci with just the right level of fitness decline would be difficult, particularly as laboratory fitness measurements may not correlate well with field measurements.

If one is targeting a homozygous lethal, then it is better if there are no heterozygous effects, though small fitness effects will not matter much. Thus, the strategy of creating homozygous lethals is robust to some level of heterozygous dysfunction. If the homozygote is sublethal, heterozygous effects can even increase the equilibrium load, but the results are enormously sensitive to fitness values which could not be accurately measured in the lab (e.g., for d=0.95, wₕₑₜ=0.53 and wₕₒᵣₙ=0.95, L=0.95, but if Wₕₑₜ=0.52, L=0). Chasing this extra load would probably not be worthwhile.

Steriles. For many species that one might want to control, killing males is a waste because it reduces the frequency of the HEG (i.e., it counts as selection against it), but will do little or nothing to reduce population growth rates or equilibrium density, which will largely be determined by female productivity. Targeting genes which, when knocked-out, lead to female sterility may therefore be more effective. That said, male sterility can be as effective as female sterility, as long as the fertilisation success of the sterile males is equal to that of the normal males (e.g., the only effect is to make sperm that are defective after karyogamy). An exception to this is for species in which females lay eggs in clusters and there is both multiple paternity and maternal sib competition; in this case sterilising males may not be as effective as sterilising females.) In principle, one might be able to engineer ‘super-sterile’ males, that sabotage female fertility. If the knock-out causes recessive sterility in both sexes, then the equilibrium frequency of the HEG is the same as for lethality (\hat{q}=e), and the frequency of sterile females is the same as the frequency of lethal females. However, the mean fitness of the population is reduced because some of the fertile females will mate with sterile males. In fact, mean fitness will be the square of what it is under lethality, because both parents have to be fertile in order for a zygote to be formed:
\overline{ŵ}=(1−e²)²

For example, with d=0.75, \overline{ŵ}=0.56; with d=0.95, \overline{ŵ}=0.036. As for lethals, some residual homozygous fertility can further decrease mean fitness.

Sex-specific lethals and steriles. Another way to avoid the ‘wastage’ of killing males would be to target a gene that, when knocked out, is a female-specific recessive lethal. sis-a, sis-b, and fs(3)100 in D. melanogaster are all reported to act in this way (Ashburner 1989: 433 Drosophila: a laboratory handbook. Cold Spring Harbor Laboratory Press, Cold Spring Harbor). This is more effective than targeting a bisexual lethal, and, for low TRD, can be more effective than targeting a bisexual sterility gene:
\overline{ŵ}=4e²/(1+3e²).

For example, with and d=0.75, \hat{q}=0.76, \overline{ŵ}=0.43; for d=0.95, \hat{q}=0.97; \overline{ŵ}=0.055. Again, some residual homozygous fertility can further increase the load. Targeting a sex-specific sterility gene would have the same effect (again, assuming any male sterility gene knockout has no effect on fertilisation success).

To assess the relative ease or difficulty of finding such genes, it is worth considering the data for D. melanogaster (reviewed by Ashburner 1989: 435):
1) Mutagenesis surveys show that recessive male sterile and recessive female sterile mutations each arise at a frequency of about 10-15% that of recessive lethals
2) Surveys of natural populations show that about 17% of major autosomes carry a recessive male sterile, about 8% carry a recessive female sterile, and about 5% carry a recessive bisexual sterile.
3) About 400 loci are thought to be able to mutate to female sterility, of which about 50-100 are exclusively required for oogenesis.

Thus, there will be many candidate loci of all the types considered thusfar, both in this species and, in all likelihood, in others. Spradling et al. (1999) Genetics 153:135-177 describe a P-element mutagenesis experiment which uncovered hundreds of recessive lethal loci, and some recessive steriles.

Sex-specific TRD and sex linkage. Thus far it has been assumed that the HEG is active in both sexes. It is possible that in some species the germlines are sufficiently different that this is difficult to engineer. Therefore, it is of interest to consider the loads achievable if TRD only occurs in one sex. In general, the effect will be to reduce the load, but not so much as to make the strategy useless. For an HEG with sex-specific TRD that causes sex-specific sterility, with the two effects occurring in the same sex, the equilibrium frequency of the HEG and the equilibrium load are the same as for the recessive lethal case with TRD in both sexes, the only difference being it will take twice as many generations to reach a given frequency and load. (i.e., for d=0.75, \hat{q}=0.5, \overline{ŵ}=0.75, t_{1,90}=34; for d=0.95, \hat{q}=0.95; \overline{ŵ}=0.19, t_{1,90}=22). If the two effects occur in opposite sexes, then the system is less effective in reducing mean fitness: for d=0.75, \hat{q}=0.47, \overline{ŵ}=0.8; for d=0.95, \hat{q}=0.71, \overline{ŵ}=0.55. By having the two effects in the same sex, one increases the effect of TRD in increasing the frequency of the HEG, because heterozygotes (where TRD occurs) have a higher fitness relative to the mean for that sex.

Targeting an X-linked sterility gene will not usually be desirable because then drive will only occur in one sex (the homogametic sex, usually females). Autosomal targets will usually be favoured, if one can get bisexual TRD; if not, then it makes no difference.

One can get high equilibrium loads if, in a male heterogametic species, one targets an X-linked male sterility gene (which, as above, has no effect on the male's fertilisation success). For example, if d=0.75 and the fitness of the hemizygous male is 0.34, the HEG will go to fixation and \overline{ŵ}=0.34; if d=0.95 and wₕₑₘ=0.06, \overline{ŵ}=0.06. However, this situation is similar to the dominant case discussed above, and depends upon intermediate fitness effects that will be difficult to measure in the lab (if sterility is complete and wₕₑₘ=0, the gene will go extinct unless d=1, in which case it will just remain at the initial frequency). Moreover, the effects will take relatively many generations to appear (for d=0.75, t_{1,90}=1319 generations for Wₕₑₘ=0.34 and 62 generations for wₕₑₘ=0.5; for d=0.95, t_{1,90}=628 generations for Wₕₑₘ=0.06 and 107 generations for Wₕₑₘ=0.1).

Maternal effects: Sometimes, phenotypes such as lethality and sterility are due not to the individual's genotype, but rather to its mother's genotype. These are maternal effect mutations. For example, there can be maternal effect lethals: homozygous females lay eggs that fail to develop. Of these, there are two classes. First, zygotes may fail to develop regardless of sperm genotype; this will behave like a recessive female sterile. Second, zygotes may be rescued if the sperm is wild-type (i.e., both the mother and the offspring have to be homozygous for the lethality to be expressed). Early examples of such genes in D. melanogaster include rudimentary (r), fused (fu), and some alleles of deep-orange (dor) (Ashburner 1989:426).

Maternal-effect lethals can also be sex-specific: homozygous daughterless (da) females produce no or few daughters, and sonless (son) and sonkiller (sok) and snl females produce no sons (Ashburner 1989: 433-4). These might also be appropriate targets for HEGs.

Finally, there are also maternal-effect bisexual sterility mutations which give the ‘grandchildless’ phenotype: homozygous females produce viable but sterile sons and daughters. Many of these in D. melanogaster have pleiotropic effects, but two that do not are agametic and gs(1)N41 (Ashburner 1989: 437). Targeting such genes can give very high loads: d=0.75, \hat{q}=0.86, \overline{ŵ}=0.18; for d=0.95, \hat{q}=0.997, \overline{ŵ}=0.0031. That is, 99.7% of output will be destroyed, and only 0.3% functional. t_{1,90}=12.

Multiple loci. To increase the load one can target multiple loci simultaneously. In the simplest case where the TRD and phenotypic effects at one locus are independent of genotype at the other locus, then the equilibrium mean fitness will be the product of the fitnesses of the two loci separately. For example, if d=0.95 at each of two recessive lethal loci, then at each one \hat{q}=0.9, and overall the population mean fitness will be \overline{ŵ}=0.19²=0.036. For unisexual sterility genes, if d=0.95, \hat{q}=0.97 at each and \overline{ŵ}=0.0031 (assuming there is some recombination between the loci) (the same as for maternal effect sterility—suggests general formula for load for this case). Thus, one can impose as large a load as one wants, by targeting ever more genes. Simulations show that it makes little difference whether recombination between the loci is 0 or ½, and even if it is 0, it makes little difference whether the HEGs are introduced in coupling or repulsion. This is because the gene conversion events act analogously to recombination to break up correlations between loci, and so the alleles end up in linkage equilibrium.

It is also possible to target loci which together make a synthetic lethal (or sterile): the individual only dies if it is homozygous for the HEG at both loci. This might occur, for example, if one targets a redundant gene pair. However, this is not considered to be a generally worthwhile strategy, though it may help in preventing unwanted transfer of the lethal effect into further populations. If there is any difference in the TRD of the two HEGs, then the one with the higher drive (or the one with the higher starting frequency) will go to fixation, and one would be left with a system analogous to a single lethal (of the HEG with the lower TRD). Even if one managed to maintain polymorphism at both loci, the load induced would still not be as great as for 2 independent lethals.

Another possibility is to target two different sites in the same locus. In this case, the double heterozygote shows the fitness effect (i.e., there is no complementation). Simulations show that if there is no recombination, then at equilibrium, of the 4 possible haploid genotypes (−−, −+, +−, ++), the ++ type is completely absent. This is also true for if there is recombination and the gene is a bisexual recessive lethal/sterile, as the double heterozygote (the only genotype in which recombination is effective) has fitness 0. Note also in this case there is no way for a single-HEG chromosome to be converted to a double-HEG chromosome, because the double heterozygote has fitness 0. (Presumably for a sex-specific sterility gene, recombination and conversion in the unaffected sex would produce the double heterozygote and the dynamics might be different.) In this system the combined frequency of the two single-HEG chromosomes will be the same as that of a single-locus system. Thus, there is no increase in load (though there may be an increase in reliability/redundancy). Note in this system the two single-HEG chromosomes have identical fitness regardless of who they are paired with, and so there will not be any frequency-dependent selection maintaining the polymorphism, and simulations show that the equilibrium frequency of the two single-HEG chromosomes is the same as that of their starting frequency, assuming equal rates or drive. Intuition suggests that if rates of drive differ, then the one with the stronger drive will drive the other extinct, and one will be back to a single-HEG situation (though it is better than the synthetic lethal case, because one ends up with the polymorphism and load caused by the better HEG, rather than the worse).

Conditional effects. One could also target a gene which is essential, but not every generation—for example, a gene required for over-wintering/diapause. Suppose, for example, there are 5 generations of no selection, followed by 1 generation of selection. Simulations for a recessive lethal show that for d=0.75 the equilibrium frequency of the HEG will be \hat{q}=0.945, resulting in a mean fitness (=1−\hat{q}²) in the selected generation of \overline{ŵ}=0.108, and for d=0.95, \hat{q}=0.99979, \overline{ŵ}=0.00042. One can compare these to 0.75⁶=0.178 and 0.19⁶=0.000047. The cumulative amount of load may not be greater, but with density dependence the effect on populations may be greater. The balance between imposing a load and selecting for a resistant allele would also be better. Note that if one had HEGs attacking 3 loci needed for diapause, \overline{ŵ}=0.00042³˜10⁻¹⁰, more than enough to wipe out any population. These numbers could be improved further by targeting a female-specific gene that makes them unable to produce diapausing progeny (see above on unisexual sterility vs lethality).

Rather than the requirement for the targeted gene varying temporally, it could also vary spatially, in which case one could eliminate the target species from a specific locale.

Sex Ratio Distortion

One might want to skew the sex ratio of a population, either as an end in itself (e.g., because one sex is particularly noxious), or as a means towards population regulation/extinction. Several possibilities are considered.

Consider first a target species in which males are XY and females are XX. Suppose one put a male-meiosis-specific endonuclease on the X chromosome which recognised and cut a sequence on the Y in a region which was not similar to any region on the X. Then the break would not be repaired (properly?), and sperm bearing the broken Y chromosome would not be functional. This would give the endonuclease-bearing X a transmission advantage (analogous to naturally occurring driving X's), and the endonuclease gene would increase in frequency even in the absence of homing (though its rate of increase would be greater if it also homed in females). This would bias the sex ratio towards females. The endonuclease gene might go to fixation, or as sperm become limiting in the population its advantage might decline, and so it could come to a stable intermediate equilibrium frequency.

This system may be useful if the males of the species are particularly harmful and requiring control; it is perhaps unlikely to lead to extinction as population productivity probably increases with the proportion of females in a population, until males are very rare indeed. If extinction is the goal, it would be better to put the endonuclease gene on the Y-chromosome. Again, it would spread because of the transmission advantage, in the absence of homing, and as it did so the sex ratio would become biased towards males. In this case there is no obvious reason (analogous to sperm becoming limiting) for the advantage to decline, and so the endonuclease-bearing Y may become fixed in the population.

What if one put an endonuclease gene targeting a X-chromosome on an autosome instead? For example, this might be necessary if the males are X0, not XY. Then the endonuclease gene would no longer have a transmission advantage, and in order to drive it through the population one would have to engineer it recognise and cut its own (empty) insertion on the autosome as well, so that it homed.

If females are ZW and males are ZZ (as in Lepidoptera, some of which are agricultural pests), then the analogous strategy would be to put the endonuclease gene on the Z, targeting the W,and thus creating a male-biased sex ratio. This would work best in species in which females lay eggs in clusters, so that females inheriting the endonuclease-bearing Z from their mothers benefit from the death of their brothers.

Another possibility would be to engineer an HEG to target a gene involved in sex determination, so that chromosomal males would be converted to females, or vice versa (i.e., make it a feminising, or masculinising, gene). This could also bias the sex ratio, though the fate of such a gene would depend upon many details of the biology of the species concerned (e.g., XX male Drosophila are sterile, because of important fertility factors on the Y, but XX males in normally X0 male species may be fertile; XY females of some species are fertile (e.g., lemmings), but not others (e.g., humans); in species like lemmings, a feminiser on the X chromosome will have a transmission advantage due to reduced competition with (lethal) YY brothers; etc.).

Resistance Alleles

If one targets a gene which, when knocked-out, is strongly deleterious, then there will be strong selection in favour of resistance alleles—sequences which are functional, but are not recognised and cut by the HEG. One could engineer these by, for example, using the degenerate property of the genetic code to create a DNA sequence which coded for the same amino acid sequence but differed from the target sequence (e.g., by changing many 3^{rd} position sites). Such resistant alleles have a number of possible uses:
1) If one has released an HEG and then wants to ‘recall’ it (e.g., because of unintended consequences), one could release individuals engineered with the resistant allele. This is shown in the lower panels of FIG. 4 for the case of an HEG causing a recessive lethal which is introduced as 1% of the population, for d=0.75 and d=0.95, with the resistant allele introduced at 1% in generation 50.
2) If one does not want to knock-out a gene throughout the population, but instead just wanted to change the sequence of the gene, one could release simultaneously individuals carrying the HEG allele and the resistant allele. This is shown in the upper panels of FIG. 4 for the same conditions, except the resistant allele is introduced simultaneously with the HEG.

In all cases, one can see that the resistant allele comes to predominate is relatively quickly after introduction.

There are many possible uses for such resistant alleles; the resistant allele may be combined in an inversion with some novel gene one wants to spread through the population. As indicated in the introduction, one would still be limited to genes that are not seriously detrimental, otherwise nonfunctional variants would spread. Nevertheless, this will often be better than attaching the gene to a transposable element or cytoplasmic element, because: (i) mutation rates will be lower than for transposable elements (because there is no transposition); (ii) one will maintain control over copy number and genomic location of the novel gene; (iii) the gene will be nuclear.

Another possible use is in creating non-interbreeding populations: in a large continuous population with local dispersal, one could release at one end an HEG and a resistance allele, and at the other end the same HEG and a resistance allele which is tied up in some chromosomal rearrangement (e.g., a translocation). The HEG would sweep through the population, followed by the resistance alleles, until they met in the middle; the two resistance genotypes would not be able to cross (because of the abnormality), and one would end up with 2 non-interbreeding gene pools. For this strategy to work one would have to release a sufficient number of hosts with chromosomal rearrangements that they could mate with each other. The numbers required would depend upon the target population density and viscosity.

Alternatively, one could release a resistant allele tied up in a chromosomal rerrangement, which also had some conditional defect.

Natural Resistance Alleles

This rapid selection for introduced resistance alleles indicates that naturally occurring resistant will also be rapidly selected, and this should be taken into account, particularly if one is targeting a strongly selected gene and trying to impose a load. Before designing one's HEG, one would first want to measure sequence variation in the target site, ideally choosing sites that show little variation. The problem is that one probably could not sequence much more than 10³ or 10⁴ alleles, and alleles at a lower frequency could not be detected directly. One would have to use some rule of thumb: e.g., make an HEG which recognises and cleaves all observed sequence variants, and all single-nucleotide differences from it. The HEG will itself diversify as it spreads through the population, by mutation, and this will help the HEG to overcome any resistant alleles that are present, as long as they are not too divergent (though perhaps to a limited extent—this coevolution could be modelled). Insertion or deletion mutations in the target site may be particularly difficult for the HEG to recognise, and so one might want to choose regions which are well conserved for length across species, or for which structural information suggests that any length variant is likely to be nonfunctional. However, if the HEGs are themselves modular, designed to recognise codons, then this might be less of a problem, as length variation in the HEG will be generated by mutation as it spreads through a population.

Though HEG function should be robust to small differences in the target site, one may not want to have it too non-specific: cleaving of nonhomologous sites will reduce the fitness of the HEG, and ideally one does not want the HEG to recognise the homologous sequence in a related species. With reference to this last point, one therefore wants to target a site which shows little variation within species, but considerable divergence between species.

If naturally occurring resistant alleles are a problem, then there are several possible solutions involving targeting multiple sites.

(i) The simplest would be to target multiple independent loci. As a worst case scenario, suppose that one is targeting recessive lethal genes and at every one there is a resistant allele at a frequency 10⁻⁴. If release frequencies are 1% and all HEGs have d=0.95, then there is a 5 generation window (from generations 10 to 14 inclusive) in which the mean fitness of the population averages about 0.3ⁿ, where n is the number of HEGs released. If one releases 20 HEGs, then mean fitness will be below 10⁻¹⁰, enough to drive any population extinct. If d=0.75, then there is a 23 generation window (from generations 15 to 37 inclusive) where mean fitness averages about 0.76ⁿ, and one would need to release 84 HEGs to get mean fitness below 10⁻¹⁰. Because of the longer window, one would not have to drive them extinct in one generation; mean fitness of 10⁻³ might well do, and this could be achieved with 25 HEGs. [One extra factor here is that if there are multiple HEGs active in the same individual, then there may be dominance effects due to all the chromosomal rearrangements.]

(ii) If the loci were tightly linked, it would take longer to select for the multiply resistant genotype.

(iii) One could target different sites in the same gene. Apart from allowing one to get even smaller rates of recombination, this also differs from the above in that the double heterozygote is lethals instead of viable.

(iv) One could make the HEGs adjacent to each other, recognising adjacent sequences. One could do this either by making two separate HEGs, or making one HEG with 2 different DNA binding domains. If either recognition site was able to be cut, then (both) HEGs would be transmitted. Since alleles resistant to only one of the HEGs would have little selective advantage (arising only from nonfunctional mutant HEGs), the evolution of the double resistant allele would be substantially retarded. The only limit on the number of adjacent HEGs one could use would be in the length of sequence that can be copied from one chromosome to another during recombinational repair.

(v) Finally, one could engineer 2 different HEGs, say A and B, attacking the same gene, and put A in the recognition site for B, and B in the recognition site for A. This would mean that the HEGs would remain together—if a -A allele is created, then it will probably be a dominant lethal if it forms a zygote with a—or -A allele (due to recurrent cutting with no resistant template for repair), or, at the very least, no spread of A, and it would recreate the BA if it was paired with a B- or BA gamete (if this was viable & fertile—this would depend upon sex-specific effects).

Problems with resistant alleles are also a reason to target a gene on the Y-chromosome using an endonuclease on the X: there is no way for a double-resistant to arise by recombination.

Other classes of resistant genotypes are also possible, though whether either one is likely to arise in any real population is unclear. First, a mutation might arise which compensates for the knock-out of the target gene but is otherwise neutral. Were such a mutation to arise, then it too would increase rapidly in frequency, and population mean fitness would return to normal. A simple duplication of the target locus is unlikely to be sufficient, as the HEG will readily transfer over to the new locus. If such resistance is a problem then it can be avoided by attacking multiple genes simultaneously.

Finally, a mutation might arise which somehow reduces or eliminates the homing activity, but is otherwise neutral. Several points here seem relevant. (I) Homing depends upon very basic cellular processes (transcription, translation, nuclear transport, recombinational repair), and so it is not clear how such a mutation might arise. (ii) Many prospective target species do not appear to have HEGs naturally, and so are unlikely to have evolved general defences against them. (iii) Naturally occurring HEGs fall into 3 or 4 distinct protein families (Chevalier & Stoddard (2001)) and artificial HEGs can be different again (eg fusions of a sequence-specific zinc finger protein with a nonspecific endonuclease domain—Bibikova et al (2001)), so if resistance evolves against one of them, there may not be cross-resistance with others. (iv) Site-specific selfish genes are also available that use reverse transcription rather than recombinational repair to propagate, including group II introns and some LINE-like transposable elements, and cross-resistance with these is highly unlikely.

Imposing a Load—Small Populations

Thus far we have been assuming that the target population is so large that the release population is constrained to be an insignificant fraction of the total (except above on chromosomal rearrangements). However, sometimes the target population is small, and this constraint does not exist, and one can release a sufficient number that, say, 90% of the population are released organisms. This is the case with all uses of the sterile insect release technique. The use of HEGs can have some advantages over release of sterile males, as the HEG can persist in the population for successive generations (depending upon the gene targeted).

An example of a small population that could be targetted is a sexually-reproducing pathogen population within a single host/patient. This may include a nematode infection. Other examples of small populations may include populations infesting an enclosed or isolated environment, for example a lake, island or greenhouse.

Though all the above theory for small releases is still correct with large releases, some of it will not matter—in particular, (i) the time to achieve a significant load is irrelevant, because there will be a load in the very first generation, and (ii) one can target a gene for which the equilibrium HEG frequency and load may be 0, but before that is reached the population will have been significantly impacted, and perhaps even driven extinct. Note a possibly desirable feature of the latter case is that a rare escapee to a different population of species would have no effect—it would just be lost (see section on ‘Restricting dispersal using dominance effects’).

One approach already used for this situation is the release of sterile males. Under ideal circumstances, if one can dilute the natural male population X-fold with sterile males, then one will reduce the population productivity X-fold; if one does this in successive generations, then in each generation this will be repeated. This is a natural basis of comparison for techniques using HEGs. [Note that in a real situation where one is driving a population extinct, one would probably release a constant number of males each generation, which would be an increasing dilution rate, as the population got smaller. However, as here we are just interested in a comparison between the two methods, this complication need not concern us.]

Perhaps the easiest situation will be if the target species is female heterogametic, in which case males are ZZ and females are ZW. One could then release males that are heterozygous for an HEG targeting a Z-linked female sterility gene; there should be relatively many such genes, as they will be hemizygous in females, and so knocking-out just one copy of the gene is more likely to have severely deleterious effects. Suppose one is able to dilute the male population X-fold every generation with males homozygous for the HEG. The equilibrium mean fitness will then be

$$\hat{\overline{w}} = \frac{1 - d}{X - d}$$

By contrast, if one dilutes the male population X-fold with sterile males (the conventional sterile male technique), then the equilibrium mean fitness is simply

$$\hat{\overline{w}} = \frac{1}{X}$$

For example, if one can dilute the population 10-fold, then the conventional sterile male technique will give a mean fitness of 0.1, whereas with the HEGs it will be 0.027 if d=0.75, or 0.0055 if d=0.95, improvements of almost 4-fold and 20-fold, respectively.

Another basis of comparison is simply to release males homozygous for a female-specific lethal/sterile, which shows Mendelian inheritance, as suggested by Thomas et al. (2000) Science 287, 2474-2476. Then the load is as given above, with d=0.5. For a 10-fold dilution, this gives a mean fitness of 0.053, only twice as good as the sterile male technique. [Curiously, Thomas et al. state it is no better than the sterile male technique.]

Note that recessive effects are useless here. If d=0.75, then mean fitness is 0.26, and if d=0.95, it is 0.050. Dominant effects are much better.

If the target species is male heterogametic, then the analogous thing would be to release homozygous females. This will add to the population productivity, and so is will usually not be desirable (unless there is substantial parental investment by males, or rates of drive are very high.) Rather, one will have to find an autosomal gene which is haplo-insufficient for female fertility (or viability) (i.e., a dominant sterile/lethal). Targeting such a gene will give the same load as above. The gene would have to be autosomal; targeting an X-linked gene would mean there was no TRD in males, and so one might as well not use an HEG.

These ideas could be combined with those in other sections (e.g., sex ratio distortion, or conditional lethals/steriles).

Restricting Dispersal Using Dominance Effects

Thusfar I have considered just a single population. Suppose there are two populations, between which there is some low level of migration, and one wants to restrict the HEG to only one population. In principle, this is possible by targeting genes that if knocked out are deleterious in the heterozygous state. This is because the effect TRD increases with the frequency of the HEG (i.e., it is positively frequency dependent and the effect of natural selection can be made to decrease with HEG). Consider first TRD. In the absence of any other force, the change in HEG frequency due to TRD is
Δq=epq
where q is the frequency of the HEG and p=1−q. Therefore, the ‘effective’ selection coefficient (i.e., the selection coefficient that would give an equivalent change in allele frequency) is:

$${s}_{e} = \frac{\frac{q + epq}{p - epq}}{\frac{q}{p}} - 1 = \frac{e}{1 - eq}$$

This increases from e to

$$\frac{e}{1 - e}$$

as q increases from 0 to 1. This positive frequency dependence means that by choosing targets with the right heterozygous effect, the fate of the HEG will depend upon its frequency, going extinct if it is very rare, but going to fixation if more common.

To be more precise, consider HEGs that are strictly dominant (i.e., the − homozygote has fitness 1 and the −+ heterozygote and ++ homozygote have fitness 1−s). The net selection coefficient for the HEG combining both the effects of drive and natural selection is then

$${s}_{net} = \frac{e - es - ( 1 - q ) s}{1 - eq ( 1 - s ) - qs}$$

This is plotted as a function of HEG frequency in the top row of FIG. 5, for different values of s and d(=(e+1)/2). Note that the frequency at which the lines cross the abscissa is the unstable equilibrium frequency. FIG. 5 shows fitness curves calculated as the integrals of the selection coefficients; a constant has been added to each so that their minimums are at 0. Populations will evolve up the curves, with the slope of the curve equal to the net selection coefficient.

Thus, in principle it is possible to target the knock-out to a single population separated from others such that there are few migrants between them each generation. However, this will require that the knock-out be deleterious in the heterozygous state, and that the HEG have just the right level of TRD.

Alternatively, an “inundative” strategy may be used for eradicating only one population whilst leaving others in the rest of the species range undisturbed. The manipulations discussed so far are “inoculative”, in that the release of relatively few engineered individuals will drive the population manipulation. This may often be an advantage, but not always; an “inoculative” strategy may not be appropriate if it is intended to eradicate one population only. “Inundative” strategies such as the release of sterile males (Knipling (1979) The basic principles of insect population suppression and management. Washington: US Department of Agriculture) are inherently self-limiting, and so more appropriate for such population-specific targeting. Engineered HEGs could be used in an inundative strategy if they were to cause dominant female lethality or sterility. Knock-outs causing dominant female-specific effects are rare, but if the HEG was engineered to be constitutively active in all tissues, then even if a zygote started heterozygous, the organism would be converted to a homozygote. Thus, one could still target a recessive female-specific locus. Females inheriting the HEG would be dead or sterile, and males would pass on the HEG to the next generation. As long as the HEG was not perfectly efficient (e<1), it would slowly disappear from the population, but could cause a substantial load before doing so. Thus, simply by changing the promoter, the threat of rare emigrants to neighbouring populations can be avoided. The use of such engineered HEGs would be more efficient than the release of sterile males, allowing either fewer individuals to be released, or larger populations to be targeted (Thomas et al (2000) Insect population control using a dominant, repressible, lethal genetic system Science 287, 2474-2476).

Introducing a Gene of Interest.

Though knocking-out a gene provides advantages, for example in stability and simplicity, HEGs may also be used to introduce novel genes into populations (much as transposable elements and cytoplasmic elements have been proposed in the past). There are two possibilities. First, the novel gene of interest could be attached to an HEG and the whole construct introduced into a recognition site in the genome, and introduced into the target population. The HEG would then spread, bringing with it the gene of interest. Alternatively, an HEG could be engineered that has more than one recognition site in the genome, and then the HEG introduced into one of them and the novel gene of interest introduced into another. Then, the HEG will spread, by cutting chromosomes not containing it; as it does so, it will also cause the gene of interest to spread by cutting chromosomes not containing it. That is, the enzyme will cut both recognition sites, and in repairing one the HEG will increase in frequency, and in repairing the other the gene of interest will increase in frequency.

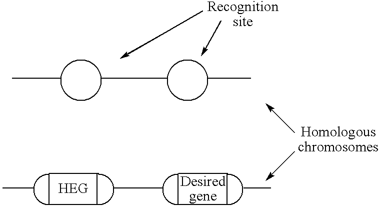

Population model: In the first case, if the inheritance of the construct is d (0≦d≦1, d=0.5 is the Mendelian rate), and we imagine a random mating population with discrete generations, and the frequency of the construct in one generation is p, then in the next generation it will be p′=p²+2p(1−p)d. In the second case, if the frequency of the HEG is p and the frequency of the gene of interest is q, then in the next generation p′=p²+2p(1−p)d (as before), and q′=q²+2q(1−q)(d(1−(1−p)′)+0.5(1−p)²). For example, if d=0.95 (for both) and both genes are introduced at 1%, then after 10 generations the HEG will be at 99% and after 16 generations the novel gene will be at 99%.

This method of using HEGs to drive genes of interest into natural populations has a number of attractive features:
(1) A conceptually simple design giving very fast drive.
(2) Better control of copy number than with transposable elements.
(3) Lower mutation rate for the gene of interest than retro-transposable elements (particularly important if there is negative selection against the gene of interest).
(4) Gene of interest is placed in the nucleus (unlike with Wolbachia).
(5) Gene of interest and HEG less likely to be transferred to another species because at no time is it separate from the host genome (unlike transposable elements and Wolbachia).
(6) In the second case, where the gene of interest is unlinked to the HEG, if the latter is transferred between species (e.g. by a virus), the other gene won't be transferred with it (unlike transposable elements and Wolbachia).
(7) The whole process should be reversible by introducing another HEG which cuts the gene of interest, but not chromosomes that do not contain it (unlike transposable elements and Wolbachia).
   Using the Methods

Techniques useful in designing HEGs to target a specific sequence are noted above, and include rational design and in vitro selection. It is also desirable to identify a gene or genes in a particular species(s) with the appropriate phenotype when knocked out and which not too variable within species, but is divergent between species. This may be done by comparisons between species and by investigating the properties of organisms in which the gene has been knocked out. The HEG may be introduced into the chosen gene using transformation techniques known to those skilled in the art. For example, a transposable element may be used, which may have to hop around until it gets to the right spot. It is preferable for the HEG to be on a nonautonomous element with transposase supplied in trans, so that the insertion is stable in the field. Alternatively, the HEG may be introduced on a plasmid from which the HEG is expressed. The HEG may then cleave the target sequence and be copied during repair. Chandrasegaran & Smith (1999) discuss the use of engineered endonucleases in methods of inserting exogenous DNA at defined sites in chromosomal DNA. Techniques described in Rong & Golic (2000) Science 288, 2013-2018 may also be useful.

Once organism(s) with the HEG inserted at the desired location have been prepared, enough organisms must be reared ready for release. Pesticide treatment (or other suitable treatment that reduces the size or fitness of the target population) just before release can help increase the fraction released.

It may also be desirable to perform demographic modelling on spread, and ecological modelling on the effect of knocking out a population of a species.

As well as the use for eradicating a population, the techniques may be used, for example, to knock out a gene important for mosquitoes to transmit malaria. The selection coefficient for such a gene might be as low as 10⁻⁶ (the mutation rate; were it lower than this, then the gene could not persist in the face of mutations), and knocking it out may have no effect on the population dynamics of mosquitoes. The HEG would spread to fixation. A drawback of this scenario is that then live HEGs are around in the community for longer, increasing the probability of jumping to a new species. An alternative approach with much the same end-point would be to use the HEGs to drive the population extinct (and the HEG extinct as well), and then release mosquitoes with the desired gene(s) missing.

Re horizontal transmission to other species, an advantage of HEGs is that they have no extra-chromosomal part of the life cycle, and no time when the protein is bound to the gene. For horizontal transmission to occur, one needs the DNA containing the HEG to somehow get into a germ-line nucleus of another species, and be sufficiently intact that it can be transcribed, and then used as a template for repair. DNA transposons and retrotransposons (which do get horizontally transmitted at some frequency) do form extra-chromosomal protein-nucleic acid complexes, and just they need to be transferred. Also, once in the new nucleus, all they have to do is insert anywhere in the new genome.

To make an HEG more species-specific, in addition to using a species-specific recognition site, one could have it be regulated by a species-specific promoter, and, in general, to be flanked by species-specific DNA. On this basis, male sterility genes might be attractive, because they evolve so quickly, but presumably there are also female-specific genes which are coevolving (though whether knocking out these gives 10% reduction in fertility or 100% is not clear).

Endonuclease Engineering

The approach depends upon engineering an endonuclease to have a new target specificity. To do so, one could start with an extant HEG. These fall into 3-4 families, and are reviewed in, for example, Mueller et al in Nucleases Eds Linn, Lloyd & Roberts, Cold String Harbor Lab Press, 2^{nd} Ed, Vol 2, pp 111-143 (1993); Belfort & Roberts (1997) Nucl Acids Res 25, 3379-3388; Dalgaard et al (1997) Nucl Acids Res 25, 4626-4638; Jurica & Stoddard (1999) Cell Mol Life Sci 55, 1304-1326.

Alternatively, the approach suggested by Chandrasegaran & Smith (1999) may be used, involving attaching a DNA cutting module onto a DNA binding module, analogously to the naturally occurring restriction enzyme Fok I. Chandrasegaran & Smith (1999) propose 3 different types of DNA binding modules: zinc fingers, helix-turn-helix and helix-loop-helix containing a leucine zipper motif The former is attractive because various rules governing recognition have been proposed and modular design is possible (see, for example, WO98/53059, WO00/27878, WO98/53057, WO96/06166, WO00/42219 and WO98/53058). Whichever the starting point for the engineering, one could then proceed by rational design, by some selection scheme, or by some combination of the two.

As an site-specific nonMendelian gene which can insert into a target site and disrupt a gene, in addition to HEGs two other classes of selfish genetic elements might also be appropriate: retrohoming group II introns and site-specific LINE-like transposable elements. Both of these move via an RNA intermediate. The retro-homing group II introns in particular may be attractive because the molecular basis of the site-specificity is better known for them than for either HEGs or LINEs, as it is based partly on RNA-DNA basepairing. However, one difficulty with them is that splicing is a necessary prelude to mobility. So, one would need to have splicing and mobility in the germ-line but not the soma, yet if one released such an element it would be susceptible to being overtaken by a mutant which splices in both germ-line and soma. Such a mutant would not impose a load on the population. Also, no nuclear group II intron has been reported, and they may be restricted to mitochondria, though Guo et al (2000) Science 289, 452-457 suggests that this may not be the case. Finally, with both alternatives there is an extrachromosomal RNA phase of the life cycle, which may make horizontal transmission more likely than for HEGs, which have no such phase, requiring direct interactions between chromosomal DNA to spread. However, none of these considerations is fatal, and these alternative possibilities may also be useful.

General
d = 0.75 (e = 0.5) d= 0.95 (e = 0.9)

| | \hat{q} | $\hat{\overline{w}}$ | \hat{q} | $\hat{\overline{W}}$ | t_{1,90} | \hat{q} | $\hat{\overline{W}}$ | t_{1,90} |
|---|---|---|---|---|---|---|---|---|
| Lethal | e | 1 − e² | 0.5 | 0.75 | 17 | 0.9 | 0.19 | 12 |
| Sublethal | $Min [ 1 , \frac{e}{s} ]$ | $1 - {sMin [ 1 , \frac{e}{s} ]}^{2}$ | 1 | 0.5 (0.5) | 36 | 1 | 0.1 (0.1) | 14 |
| Bisexual sterility | e | (1 − e²)² | 0.5 | 0.5625 | 17 | 0.9 | 0.0361 | 11 |
| Bisexual substerility | $Min [ 1 , \frac{e}{s} ]$ | ${( 1 - {sMin [ 1 , \frac{e}{s} ]}^{2} )}^{2}$ | 1 | 0.25 (0.5) | 30 | 1 | 0.01 (0.1) | 11 |
| Unisexual sterility | $\frac{2 e + 2 {e}^{2} + 4 {e}^{3}}{( 1 + e ) ( 1 + 3 {e}^{2} )}$ | $\frac{4 {e}^{2}}{1 + 3 {e}^{2}}$ | 0.76 | 0.43 | 18 | 0.97 | 0.055 | 11 |
| Unisexual substerility | $Min [ 1 , \frac{2 e ( e ( 2 e + s ) + s )}{s ( 1 + e ) ( {e}^{2} ( 4 - s ) + s )} ]$ | $1 - sMin [ 1 , \frac{4 {e}^{2}}{s ( {e}^{2} ( 4 - s ) + s )} ]$ | 1 | 0.33 (0.33) | 26 | 1 | 0.053 (0.053) | 11 |
| Unisexual drive & | e | 1 − e² | 0.5 | 0.75 | 34 | 0.9 | 0.19 | 22 |
| sterility, same sex* | | | | | | | | |
| Unisexual drive & substerility, same sex* | $Min [ 1 , \frac{e}{s} ]$ | $1 - {sMin [ 1 , \frac{e}{s} ]}^{2}$ | 1 | 0.5 (0.5) | 73 | 1 | 0.1(0.1) | 26 |
| Unisexual drive & sterility, opposite sex | $\frac{e + {e}^{2} + {e}^{3}}{1 + e + {e}^{2} + {e}^{3}}$ | $\frac{1}{1 + {e}^{2}}$ | 0.47 | 0.8 | 32 | 0.71 | 0.55 | 19 |
| Unisexual drive & substerility, opposite sex | $Min [ 1 , \frac{e ( s + e ( s + 3 ) )}{s ( e + 1 ) ( {e}^{2} ( 2 - s ) + s )} ]$ | $1 - sMin [ 1 , \frac{{e}^{2}}{s ( {e}^{2} ( 2 - s ) + s )} ]$ | 1 | 0.67 (0.67) | 64 | 1 | 0.53 (0.53) | 27 |
| Maternal effect | | | 0.86 | 0.18 | 20 | 0.997 | 0.0031 | 12 |
| bisexual sterility | | | | | | | | |
| Maternal effect | | | 0.998 | 0.11 | 24 | 0.9999 | 0.0028 | 12 |
| bisexual substerility | | | | (0.33) | | | (0.05) | |
| Two lethals | | | 0.5 | 0.56 | | 0.9 | 0.036 | |
| Two unisexual | | | 0.76 | 0.18 | | 0.97 | 0.0031 | |
| steriles | | | | | | | | |
| Two synthetic | | | 0.81 | 0.59 | | 0.97 | 0.13 | |
| lethal† | | | | | | | | |

*Same as X-linked female sterility gene in male heterogametic species.
†Requires (unrealistic) assumption of equal drive at the two loci.

Practical Steps for Implementation

1. Identify an appropriate species and class of gene to be targeted. For example, to control malaria, one must decide whether to target the malaria parasite or the mosquito vector. If the mosquito, does one try to eliminate them, or transform them so that they are no longer able to transmit malaria? An ecological assessment may be made of the wider community/ecosystem ramifications of the proposed population engineering.

2. Identify a target gene. This will depend upon what one wants to do and what is already known about the organism. If one wants to target a female sterility gene in Drosophila melanogaster for reasons of population control, there are dozens of female sterility genes already known, and one would just look in the literature (e.g., flybase). If one wanted to target a female sterility gene in mosquitoes, one could (i) look for genes homologous to female sterility genes in Drosophila (e.g., by PCR with degenerate primers, or searching the mosquito genome information vailable in databases); or (ii) look for genes that are expressed only in ovaries e.g., by cDNA libraries, or microarray experiments); or (iii) do a mutational screen for such genes, and the clone the mutant. If one wants to disrupt a gene necessary for mosquitoes to transmit malaria, one could look for candidates in the interaction between malaria and mosquito, or do a mutational screen. Steps would be similar if one wanted to target a diapause-specific gene, or a maternal-effect gene.

3. Identify recognition and insertion sites in the target gene. The recognition site may be 15-40 bp, and the insertion site will be in the middle of it. A random 15-40 bp in the target gene, may be chosen, or further selection techniques used. For example, multiple alleles from the target population may be sequenced, in order to choose a region with low sequence diversity. Functional studies (e.g., site-directed mutagenesis studies, and/or structural studies), may be performed in order to identify regions likely to be conserved within the population. The same gene may be sequenced in related species, and regions chosen which differ from the target population, in order to reduce the probability of the homing endonuclease escaping to another species. It may also be useful to check to confirm that the chosen recognition sequence exists only once in the genome. It is also highly desirable to confirm that insertion of the HEG into the chosen site actually disrupts the function of the gene.

4. Engineer an endonuclease to recognise the chosen sequence. This may be done by a combination of rational design and selection (either in vitro or in some simple model organism). As starting point, an existing homing endonuclease gene may be taken and its protein sequence altered to recognise the desired sequence. Alternatively, a known DNA binding protein may be added to a DNA endonuclease domain. Chandrasegaran & Smith (1999), and other references as discussed above, discuss in more detail how this can be done. If using a group II intron, then Guo et al (2000) demonstrates how the sequence specificity may be altered. It may be useful to confirm at this point that the endonuclease does not recognise the homologous sequence in related species. It may also be useful to design a resistant sequence, which is still functional in the host species, but is not recognised by the HEG (e.g., by changing all the synonomous sites), to have as backup in case the population engineering is to be aborted.

5. Put the endonuclease under the control of a germ-line-specific promoter. Such a promoter may be identified by searching for genes showing germ-line-specific expression in the target species (e.g., in the literature, or by homology to genes in the literature, or by cDNA library/microarray experiments), and identifying the promoter for such a gene. For example, one could use the promoter of a meiosis-specific gene (e.g., spo11 and/or spo13 in the yeast Saccharomyces cerevisiae, or homologues in other species).

6. Introduce the HEG into the correct position in the host genome. For some species methods for doing this are already known (e.g., yeast, mice). In other species, introducing HEGs at the correct site may be easier than other genes, because if a plasmid carrying the target gene and HEG is introduced into the cell and expressed, then the target gene on the host chromosome will be cut, and repaired using the plasmid.

7. Introducing engineered hosts into the natural population. This is simply a matter of growing up as many of the engineered hosts as is feasible and releasing them. This may be done either at a time of the year when the natural population is low (for some species, in spring), or after pesticide treatment (or other population-reducing or weakening treatment), so that the released individuals start as a higher percentage, but this is not necessary.

8. Monitor the spread of the HEG by collecting individuals and scoring their genotype (e.g., by PCR).

### EXAMPLE 2

### Outcrossing Sex Allows a Selfish Gene to Invade Yeast Populations.

Homing endonuclease genes in eukaryotes are optional genes that have no obvious effect on host phenotype except causing chromosomes not containing a copy of the gene to be cut, thus causing them to be inherited at a greater than Mendelian rate via gene conversion. These genes are therefore expected to increase in frequency in outcrossed populations, but not in obligately selfed populations. To test this idea, we compared the dynamics of the VDE homing endonuclease gene in six replicate outcrossed and inbred populations of yeast (S. cerevisiae). VDE increased in frequency from 0.21 to 0.55 in four outcrossed generations, but showed no change in frequency in the inbred populations. The absence of change in the inbred populations indicates that any effect of VDE on mitotic replication rates is less than 1%. Data from the outcrossed populations best fits a model in which 82% of individuals are derived from outcrossing and VDE is inherited by 74% of the meiotic products from heterozygotes (compared to 50% for Mendelian genes). These results demonstrate empirically how host mating system plays a key role in determining the population dynamics of a selfish gene.

Phylogenetic surveys show that HEGs are sporadically distributed among closely related species, and sequence analysis indicates some of these HEGs contain frameshift mutations and are presumably non-functional (Goddard & Burt 1999). There is also strong phylogenetic evidence for the rampant horizontal transmission of HEGs (Goddard & Burt 1999; Koufopanou et al. 2001; Vaughn et al. 1995). These observations led to the construction of a cyclical model of HEG evolution which suggests that horizontal transfer events introduce HEGs to uninfected populations, and this is followed by HEG population invasion, then degeneration, and finally loss; the cycle may be initiated once more with another HEG horizontal transfer event (Goddard & Burt 1999). Frequent horizontal transfer may allow HEGs to persist over evolutionary time by the recurrent invasion of new species or populations. One critical assumption of this model is that a newly introduced HEG will indeed spread to fixation. In the absence of any countervailing forces, the transmission ratio distortion shown by an HEG will lead to it increasing in frequency in an outcrossed sexual population. However, this has never been empirically demonstrated, and may be prevented if, for example, the gene substantially reduces host fitness. Moreover, all else being equal, HEGs should not increase in frequency in a wholly inbred population since gametes from independent HEG⁺ and HEG⁻ lineages are not brought together and provide no opportunity for super-Mendelian inheritance. HEGs may only increase within inbred populations if they confer a benefit, or if there is an extremely high rate of horizontal transfer among lineages [an upper bound estimate for HEG horizontal transfer rate encompasses infinity (Goddard & Burt 1999; Koufopanou et al. 2001)]. Previous studies in yeast have shown that other types of selfish element, in particular the 2 μm plasmid (Futcher et al. 1988) and Ty3 element (Zeyl et al. 1996), can indeed increase in frequency in sexually outcrossed populations but not in inbred ones. As yet, there are no similar data concerning HEG population dynamics.

VDE is one of the best studied HEGs and infects the middle of the metabolically important VMA1 gene (which codes for a sub-unit of the vacuolar ATP pump) (Gimble & Thorner 1992). Ordinarily an insertion within VMA1 should destroy its function. However, the ATP pump sub-unit derived from VDE⁺ alleles is not compromised since VDE self-splices at the protein level (Chong et al. 1996) to leave a functionally intact VMA1p and the free VDE protein product PI-SceI (see FIG. 3); such elements are known as inteins (Colston & Davis 1994). PI-SceI has an endonuclease function: it uniquely recognises VMA1 alleles which do not contain VDE, and cuts them at the exact point where VDE is inserted in VDE⁺ alleles (Gimble & Wang 1996). This break initiates the cells repair pathway which results in the conversion of VDE alleles and thus facilitates VDE's super-Mendelian inheritance. We test the validity of ideas concerning the HEGs capability for population invasion by comparing VDE's change in frequency between experimentally inbred and outcrossed populations of Saccharomyces cerevisiae.

Materials and Methods

Strains and Microbiological Methods

We constructed isogenic VDE⁺ and VDE⁻ strains of Saccharomyces cerevisiae by transforming the haploids DH89α and DH90a ho, ura3 (descendants of the wild type Y55) with the YEpVMA1 plasmid (Gimble & Thorner 1993), which contains the VDE⁺ allele. These haploids were mated and put through meiosis which allowed VDE to home into the genomic VDE⁻ allele; the resulting VDE⁺ strains DH91 and 95 were subsequently cured of the plasmid. The two pairs of haploids were then mated to form homozygous diploids (DH89/90 and DH 91/95). These were used to found six populations which contained the VDE⁺ allele at an average frequency of 0.21. Each population was divided in two to initiate replicate inbred and outcrossed lines (see Table 1). The populations were then starved of nitrogen by placing on 2% potassium acetate and were thus stimulated to go through meiosis and produce four haploid spores (Burke et al. 2000). Yeast spores may be one of two mating types, either a or a, and gametes will only mate with an opposite mating type (though haploids may divide mitotically if no opposite mating type is encountered) (Burke et al. 2000). These four yeast spores are contained within an ascus (sac), and under normal conditions will germinate when placed on YPD (1% yeast extract, 2% peptone, 2% glucose), mate with their ascus partners and, therefore, inbreed. To facilitate outcrossing asci were broken apart by digestion with 4.4 mg/mL sulfatase (Sigma No. S9626) overnight at 30° C. and then mildly sonicated to fully dissociate the asci and randomise the spores. The spores were allowed to mate randomly and then grow by placing on YPD for roughly 15 hours at 30° C. (this equates to a maximum of 10 mitotic generations) before the next round of sporulation. Cells were removed from YPD and washed with water before again placing on 2% potassium acetate. Both inbred and outcrossed replicate populations simultaneously passed through five sexual (meiotic) generations. Since all individuals started homozygous, the opportunity for VDE to display super-Mendelian inheritance only arose in generations 2-5, once heterozygous genotypes were produced.

For the purposes of comparison we also measured the frequency of VDE in the meiotic products arising from VDE⁺/VDE⁻ heterozygotes. Spores from 24 tetrads were dissected, allowed to form haploid colonies, and then scored for the presence/absence of VDE.

TABLE 1
Frequencies of VDE homozygous (+/+), heterozygous (+/−), and VDE free
(−/−) genotypes in each of the replicate inbred and outcrossed populations
estimated from the 95 individuals sampled from each population at each time point
using the colony PCR method. The number of individuals disregarded, because
they were determined to be haploid, are indicated in the columns headed in. The
estimated frequency of VDE is shown for each population at each time point in the
column headed p; inbreeding coefficients are shown in column headed F.

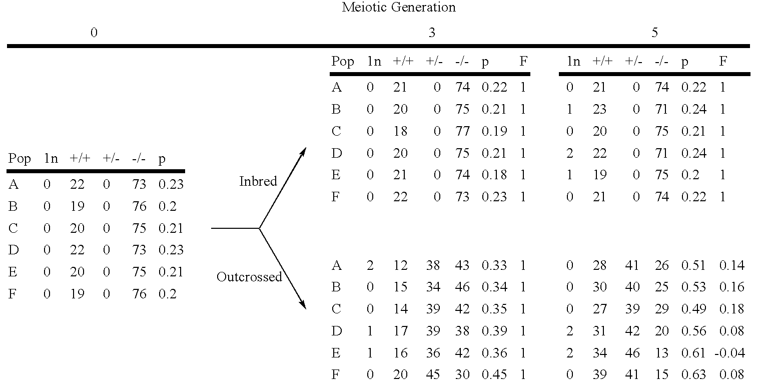

Molecular Methods

The frequency of VDE was determined at generations 0, 3 and 5 by colony PCR. Samples from each population were plated at low density on YPD and 95 colonies were picked randomly. The ploidy of these samples was determined by transferring them to sporulation medium, waiting five days, exposing them to ether vapours (Rockmill et al. 1991), and then replica plating to YPD. Only diploids would be able to make spores that would survive the ether treatment. Less than 1% of the sample colonies did not sporulate; they were presumably unmated haploids (Table 1). Original sample colonies were then suspended in 1×PCR buffer, incubated at 98° C. for 5 minutes, and then vortexed and centrifuged at 13,000 rpm for 2 minutes. 10 μL of supernatant was used as a template in a PCR reaction with primers VMA105 (5′-CAAGTACTCCAATTCTGAC) and VMA102 (5′-ATTCCATCAAGACTTCTGC) which flank the VDE insertion site. The sizes of PCR products indicated the VDE status of each yeast colony: small amplicon indicated a VDE⁻ allele (80 bp); large amplicon indicated a VDE⁺ allele (1448 bp); two amplicons indicated a heterozygote. The PCR products were electrophoresed through 1.5% agarose to determine size. The 95 PCR reactions for each sample point were performed using a 96 well plate; the 96^{th} well contained a known VDE⁺/VDE⁻ heterozygote as positive control. Since VDE only homes during meiosis and not during mitosis (Gimble & Thorner 1993), we used the frequency of VDE⁺/VDE⁻ heterozygotes to estimate outcrossing efficiency.

Results

The extent of inbreeding experienced by each of the populations was determined by calculating Wright's inbreeding coefficient (F) (Table 1), which estimates the deviation of a population from Hardy-Weinberg equilibrium (Weir 1996). The mean inbreeding coefficients for the outcrossed and inbred populations were 0.11±0.019 (se) and 1.0±0, respectively, demonstrating that asci digestion and spore randomisation were effective at producing largely outcrossed populations. The frequency of VDE increased significantly in the outcrossed populations, but not in the inbred populations (p<0.001 and p=0.34, respectively, paired t-tests of arc-sine transformed initial and final frequencies; FIG. 2). This dependency of gene frequency change on breeding system indicates that the increase is a result of super-Mendelian inheritance, and not selection for cells that are VDE⁺.

If we assume that the inbred populations are completely inbred (and the failure to detect even a single heterozygote suggests this is not too far wrong), then the change in frequency of VDE in these populations can be used to estimate the selection coefficient associated with VDE. The fact that initial and final frequencies did not differ significantly means the selection coefficient is not significantly different form zero. To put bounds on the selection coefficient we calculated the regression of ln(p/1−p) on the number of mitotic generations, assuming 10 mitotic generations per meiotic generation. The mean regression coefficient across the six replicate populations is 0.0009±0.00099 (s.e.). Our value of 10 mitotic generations per meiotic generation is an upper limit; if instead we assume there were five mitotic generations, then the selection coefficient is 0.002±0.0020. It appears that any effect VDE may have on cell replication rates, either positive or negative, is small (<1%) (at least in our experimental environment). VDE's capability for population invasion will therefore principally depend on the rate of inheritance in heterozygotes and on the frequency of outcrossing.

To analyse in more detail the spread of VDE through the outcrossed populations, we constructed a model of a selectively neutral super-Mendelian gene in a population with a mixed mating system, in which a fraction t of zygotes are produced by outcrossing and a fraction 1−t from intra-ascus selfing (see Appendix). If the frequencies of VDE⁺/VDE⁺, VDE⁺/VDE⁻, and VDE⁻/VDE⁻ individuals in one generation are x, y and z, then their frequencies in the next generation will be:
x′=t(x+dy)²+(1−t)[x+1/3(2d(d+1)−1)y]
y′2t[1−(x+dy)](x+dy)+⅔(1−t)(1−d)(2d+1)y (1)
z′=t(x+dy−1)²+(1−t)[⅔(d−1)²y+z]
where d is the frequency of VDE in the meiotic products of VDE⁺/VDE⁻ heterozygotes (d=0.5 being Mendelian inheritance). This model accounts for the fact that intra-ascus mating leads to a reduction in heterozygosity of ⅓ every generation; this differs from cases in which selfing involves fusion of gametes from independent meiosis, where heterozygosity is decreased by ½ every generation (Falconer 1981). We used this model to calculate best estimates for d and t by maximising the likelihood of obtaining the observed data assuming a multinomial distribution (Edwards 1972). The maximum likelihood estimates were d=74% (72.1% to 75.3%) and t=82% (78.2% to 87.3%) (95% confidence intervals obtained from bootstrap datasets). VDE's rate of inheritance was also estimated independently by sporulating a heterozygous strain and genotyping the spores directly. Of 96 spores, 76 were VDE⁺, giving d=79% (95% C.I. 70%-86%, calculated using the binomial distribution from Rohlf & Sokal (1995)). This alternative estimate agrees very well with the value of d from the experimental populations.
Discussion

Deviations from Mendelian inheritance can only affect population gene frequencies to the extent which the population contains heterozygotes. The frequency of heterozygotes is greater with outcrossing than with inbreeding. Therefore, one can assess the role of super-Mendelian inheritance in gene frequency changes by comparing those changes in inbred and outcrossed populations. Futcher et al. (1988) were the first to use this approach, in their study of the yeast 2 μm plasmid. Breeding system was manipulated in much the same way as was done here, though they had no independent means of estimating actual outcrossing rates under the two treatments. In two replicate outcrossed populations the plasmid increased in frequency from 0.1 to 0.4 in four sexual generations, but in the inbred populations there was no change in frequency. They concluded that the increase in frequency in the outcrossed populations was due to super-Mendelian inheritance, consistent with the plasmid's behaviour in defined crosses. In separate experiments, they estimated a cost of the plasmid for mitotic replication rates of about 1%. In our experiments on VDE, we also observed an increase in frequency in outcrossed populations but not inbred populations, and conclude that the increase in the outcrossed populations is due to super-Mendelian inheritance. Our experiments were of sufficient size that the absence of a change in the inbred populations indicates a small effect (positive or negative) of VDE on mitotic replication rates, certainly less than 1%. These results also show that horizontal transmission of VDE is not so rampant as to have a detectable effect on population dynamics in the lab.

This logic of outcrossed sexuality facilitating the spread of super-Mendelian elements is also thought to underlie certain facts on the comparative distribution of selfish genetic elements, in particular that B-chromosomes are more common in outcrossed species of plants than in inbred species (Burt & Trivers 1998), and retrotransposable elements are present in most animals except bdelloid rotifers, which are putatively anciently asexual (Arkhipova & Meselson 2000).

The strength of super-Mendelian inheritance in favour of a gene can be quantified by d, the fraction of haploid meiotic products produced by heterozygotes that carry the gene. The best estimate from our experimental populations was d=74%, similar to that derived from dissecting tetrads (79%). These values are slightly lower that the 90% (95% CI 83%-95%) reported by Ginble & Thomer (1992), derived from dissecting tetrads. This difference may be due to genetic background effects, as the strains used in the two studies were different. Nevertheless, even the lower values are such that VDE can dramatically increase in frequency over evolutionarily trivial time scales. In our experimental populations VDE increased from 21% to 56% in just four outcrossed generations, and in a fully outcrossed population it is predicted to increase in frequency from 0.1% to 99.9% in 29 generations (calculated by iterating equations 1).

These predictions apply to a fully outcrossed population, and to the extent that natural yeast populations are inbred, these times will increase. Molecular analysis of a natural population of S. paradoxus (an undomesticated close relative of S. cerevisiae) shows strong deviations from Hardy-Weinberg proportions, with an inbreeding coefficient of F=0.99 (L. Johnson, pers. comm.) This high level of inbreeding will retard the spread of a selectively neutral super-Mendelian gene 100-fold. It will also lower the maximum cost a selfish gene can impose on its host and still spread through a population. S. cerevisiae is host to a diverse community of selfish genetic elements: in addition to VDE and the 2 μm plasmid, there are also seven mitochondrial HEGs (and five more group I introns that probably once had them) (Lambowitz & Belfort 1993), two retro-homing group II introns (Bonen & Vogel 2001), five retrotransposable element families, four RNA viruses and associated satellites (which, despite the name, are vertically inherited and not infectious) (Wickner 1992), and two self-propagating prion protein conformations (Wickner et al. 1996). Notably absent from this list are DNA transposons and LINE-like retrotransposons. Perhaps these are too costly to invade such highly inbred populations.

The high frequencies of inbreeding in S. paradoxus are presumably due both to intra-ascus mating and to mating-type switching, a molecular mechanism which allows cells to mate with genetically identical clonemates (Takahashi et al. 1958). Mating-type switching proceeds by a directed gene conversion event, and the key gene involved (HO) encodes a site-specific endonuclease derived from VDE (Dalgaard et al. 1997). It seems that increased inbreeding has evolved by the domestication of an HEG, an event which, perversely, makes the spread of HEGs and other selfish genes more difficult.

Appendix

Dynamics of a super-Mendelian gene in a yeast population with mixed mating system.

We want to calculate the frequencies of VDE⁺/VDE⁺, VDE⁺/VDE⁻, and VDE⁻/VDE⁻ individuals in one generation, given that their frequencies in the previous generation were x, y and z. Recall that VDE alleles are only converted to VDE⁺ alleles during meiosis. First consider the individuals derived from random mating.

The frequency of VDE in the gametes will be u=x+yd, where d is the frequency of VDE in the meiotic products of VDE⁺/VDE⁻ heterozygotes (d=0.5 being Mendelian inheritance). The frequency of the three genotypes among outcrossed zygotes will then be u², 2u(1-u), and (1-u)², respectively. Now consider the individuals derived from intra-ascus mating. Tetrads derived from homozygous parents will give rise to homozygous offspring. Tetrads derived from heterozygous parents will have 2, 3, or 4 haploid spores that are VDE⁺, with the remainder being VDE⁻. Assuming independent conversion the two VDE⁻ alleles the relative frequency of these 3, tetrad types will be (1−c)², 2c(1−c), and c², where c=2d−1 is the probability that a particular VDE⁻ allele is converted to a VDE⁺ allele (c=0 being Mendelian inheritance). Relative frequencies of the three diploid genotypes are then calculated by assuming that the meiotic products are randomly ordered in the tetrad and that random mating occurs between spores within a tetrad. Overall, the frequencies of the three genotypes will be:
x′=tu²+(1−t){x+y[(1−c)²(⅙)+2c(1−c)(½)+c²]}
y′=2tu(1−u)+(1−t)y[(1−c)²(⅔)+2c(1−c)(½)]
z′=t(1−u)²+(1−t){z+y[(1−c)²(⅙)]},
which simplify to equations (1).
References

Arkhipova, I. & Meselson, M. 2000 Transposable elements in sexual and ancient asexual taxa. Proc. Natl. Acad. Sci. USA 97, 14473-14477.

Belfort, M. & Roberts, R. J. 1997 Homing endonucleases: keeping the house in order. Nucleic Acids Res. 25, 3379-3388.

Bonen, L. & Vogel, J. 2001 The ins and outs of group II introns. Trends in Genetics 117, 322-331.

Burke, D., Dawson, D. & Stearns, T. 2000 Methods in yeast genetics. A cold spring harbor laboratory course manual: Cold Spring Harbor Press.

Burt, A. & Trivers, R. 1998 Selfish DNA and breeding systems in flowering plants. Proc. R. Soc. Lond. B. 265, 141-146.

Colaiacovo, M. P., Paques, F. & Haber, J. E. 1999 Removal of one nonhomologous DNA end during gene conversion by a RAD1- and MSH2-independent pathway. Genetics 151, 1409-1423.

Colston, M. J. & Davies, E. O. 1994 The ins and outs of protein splicing elements. Mol. Micro. 12, 359-363.

Dalgaard, J. Z., Klar, A. J., Moser, M. J., Holley, W. R. & Chatterjee, A. 1997 Statistical modeling and analysis of the LAGLIDADG family of site-specific endonucleases and identification of an intein that encodes a site-specific endonuclease of the NHN family. Nucleic Acids Res. 25, 4626-4638.

Edwards, A. W. F. 1972 Likelihood. London: The John Hopkins university Press.

Falconer, D. S. 1981 Introduction to quantitative genetics. London: Longman.

Futcher, B., Reid, E. & Hickey, D. 1988 Maintenance of the 2 νm circle plasmid of Saccharomyces cerevisiae by sexual transmission: an example of selfish DNA. Genetics 118, 411-415.

Gimble, F. S. & Thorner, J. 1992 Homing of a DNA endonuclease gene by meiotic conversion in Saccharomyces cerevisiae. Nature 357, 301-305.

Gimble, F. S. & Thorner, J. 1993 Purification and characterisation of VDE, a site specific endonuclease from the yeast Saccharomyces cerevisiae. J. Biol. Chem. 268, 21844-21853.

Goddard, M. R. & Burt, A. 1999 Recurrent invasion and extinction of a selfish gene. Proc. Natl. Acad. Sci. USA 96, 13880-13885.

Hickey, D. A. 1982 Selfish DNA: a sexually transmitted nuclear parasite. Genetics 101, 519-531.

Koufopanou, V., Goddard, M. & Burt, A. 2001 Adaptation for horizontal transfer in a homing endonuclease. In prep.

Lambowitz, A. & Belfort, M. 1993 Introns as mobile genetic elements. Ann. Rev. Biochem. 62, 587-622.

Mueller, J., Bryk, M., Loizos, N. & Belfort, M. 1993 Homing Endonucleases. In Nucleases 2nd Ed, vol. 2 (ed. S. M. Linn, R. S. Lloyd & R. J. Roberts), pp. 111-143. Cold Spring Harbour: Cold Spring Harbour Press.

Rockmill, B., Lambie, E. J. & Roeder, G. S. 1991 Spore enrichment. In Guide to yeast genetics and molecular biology, vol. 194 (ed. C. Guthrie & G. R. Fink), pp. 146-149. London: Academic Press Ltd.

Rohlf, F. J. & Sokal, R. R. 1995 Statistical Tables: W H Freeman.

Szostak, J. W., Orr-Weaver, T. L. & Rothstein, R. J. 1983 The double-strand-break repair model for recombination. Cell 33, 25-35.

Takahashi, T., Saito, H. & Ikeda, Y. 1958 Heterothallic behaviour of a homothallic strain in Saccharomyces cerevisiae. Genetics 43, 249-260.

Vaughn, J. C., Mason, M. T., Sper-Whitis, G. L., Kuhlman, P. & Palmer, J. D. 1995 Fungal origin by horizontal transfer of a plant mitochondrial group I intron in the chimeric CoxI gene of Peperomia. Mol. Evol 41, 563-572.

Weir, B. 1996 Genetic Data Analysis II: Sinauer.

Wickner, R. B. 1992 Double-stranded and single-stranded RNA viruses of Saccharomyces cerevisiae. Annu. Rev. Microbiol. 46, 347-375.

Wickner, R. B., Masison, D. C. & Edskes, H. K. 1996 [URE3] and [PSI] as prions of Saccharomyces cerevisiae: genetic evidence and biochemical properties. Seminars in Virology 7, 215-223.

Zeyl, C., Bell, G. & Green, D. M. 1996 Sex and the spread of retrotransposon Ty3 in experimental populations of Saccharomyces cerevisiae. Genetics 143, 1567-1577.

1. A method for genetically modifying a target population of an organism, comprising the steps of
providing a modified organism, wherein the modified organism is capable of sexually reproducing with an organism of the target population, and wherein a selected gene in the germline of the modified organism is disrupted by having inserted into it a sequence-specific nonMendelian selfish gene; and
introducing the modified organism into the target population.

2. The method of claim 1 wherein the step of providing a modified organism comprises the step of preparing a modified organism by disrupting a selected gene in the germline of an organism which is capable of sexually reproducing with an organism of the target population, by inserting into the selected gene a sequence specific nonMendelian selfish gene.

3. (canceled)

4. (canceled)

5. (canceled)

6. (canceled)

7. (canceled)

8. The method of claim 1 wherein the number of modified organisms introduced into the population is sufficient for the gene disruption to spread through at least 50, 60, 70, 80, 90 or 95% of the population after about 10 to 100 generations, or for at least 50, 60, 70, 80 90 or 95% of the initial population to be eradicated or predicted to be eradicated after about 10 to 100 generations.

9. The method of claim 1 wherein the sequence-specific nonMendelian selfish gene is a homing endonuclease gene (HEG).

10. The method of claim 9 wherein the endonuclease encoded by the HEG is a recombinant endonuclease.

11. The method of claim 10 wherein the endonuclease comprises a zinc-finger DNA binding domain.

12. The method of claim 10 wherein the endonuclease comprises a non-specific DNA cleavage domain.

13. The method of claim 1 wherein the sequence-specific nonMendelian selfish gene is a retrohoming group II intron or a site-specific LINE-like transposable element.

14. The method of claim 1 wherein the sequence-specific nonMendelian selfish gene is germ-line-specific.

15. The method of claim 9 wherein expression of the endonuclease encoded by the HEG is under the control of a germ-line- or meiosis-specific promoter.

16. The method of claim 1 wherein the disrupted gene is a recessive lethal gene or a recessive sterile gene.

17. The method of claim 1 wherein the disrupted gene has the effect of producing female sterility.

18. The method of claims 1 wherein the disrupted gene is partially recessive.

19. (canceled)

20. The method of 1 wherein the germline of the modified organism further comprises an allele of the selected gene which is modified so that it does not contain a recognition site for the sequence-specific nonMendelian selfish gene; or wherein a second modified organism, the germline of which comprises an allele of the selected gene which is modified so that it does not contain a recognition site for the sequence-specific nonMendelian selfish gene, is introduced into the population.

21. The method claim 1 further comprising the steps of
providing a second modified organism, wherein the modified organism is capable of sexually reproducing with an organism of the target population, and wherein the selected gene in the germline of the modified organism is modified so that it does not contain a recognition site for the sequence-specific nonMendelian selfish gene; and
introducing the second modified organism into the target population.

22. A method for genetically modifying a target population of an organism, comprising the steps of
Introducing a homing endonuclease gene (HEG) into the germline of an organism which is capable of sexually reproducing with organisms of the target population; and
Introducing the modified organism into the target population.

23. A method according to claim 22 wherein the HEG is introduced together with a gene (foreign gene) for introduction into the target population.

24. The method of claim 23 wherein the HEG and foreign gene are inserted at different sites in the organism's genome.

25. A polynucleotide comprising a polynucleotide sequence encoding a recombinant sequence-specific endonuclease flanked by the recognition site for the said sequence-specific endonuclease such that the coding sequence for the endonuclease is inserted at the point in the recognition sequence at which the endonuclease cleaves.

26. A polynucleotide according to claim 25 wherein the endonuclease comprises a zinc-finger DNA binding domain or a non-specific DNA cleavage domain.

27. (canceled)

28. (canceled)

29. (canceled)

30. (canceled)

31. A method for altering the sex ratio in a population of an organism, comprising the steps of
Providing a modified organism, wherein the modified organism is capable of sexually reproducing with an organism of the target population, and wherein the modified organism comprises a recombinant polynucleotide encoding and capable of expressing a sequence-specific endonuclease which is capable of cleaving a sequence on a sex chromosome; and
introducing the modified organism into the target population.

32. A method for altering the sex ratio; in a population of an organism, comprising the steps of
1. Providing a modified organism, wherein the modified organism is capable of sexually reproducing with an organism of the target population, and wherein the modified organism comprises a sequence-specific nonMendelian gene targeting a DNA sequence affecting the segregation or viability of a sex chromosome; and
2. introducing the modified organism into the target population.

33. (canceled)

34. (canceled)

35. (canceled)

36. (canceled)
